SEQUENCE LISTING (6 sequences)

SEQ ID NO: 1 - DNA, 59 bp, Artificial Sequence
  Description of Artificial Sequence Synthetic oligonucleotide
tttttttttttagccaaggtaaccagtacaaggtgctaaacgtaatggcttcggcttac

SEQ ID NO: 2 - DNA, 25 bp, Artificial Sequence
  Description of Artificial Sequence Synthetic oligonucleotide
ttttttttttggttggtgtggttgg

SEQ ID NO: 3 - DNA, 25 bp, Artificial Sequence
  Description of Artificial Sequence Synthetic oligonucleotide
ttttttttttggtggtggttgtggt

SEQ ID NO: 4 - DNA, 47 bp, Artificial Sequence
  Description of Artificial Sequence Synthetic oligonucleotide
ttttttttttggggcacgtttatccgtccctcctagtggcgtgcccc

SEQ ID NO: 5 - DNA, 55 bp, Artificial Sequence
  Description of Artificial Sequence Synthetic oligonucleotide
ttttttttttgcgcggggcacgtttatccgtccctcctagtggcgtgccccgcgc

SEQ ID NO: 6 - DNA, 55 bp, Artificial Sequence
  Description of Artificial Sequence Synthetic oligonucleotide
ttttttttttctgctcgttggctgaggccgtctcgcgtcgcagcgctacggcccc

(19) United States
(12) Patent Application Publication
(10) Pub. No.: US 2017/0267716 A1
(43) Pub. Date: Sep. 21, 2017

(54) PRECIPITATION OF POLYPEPTIDES BY APTAMER-POLYMER CONJUGATES

(71) Applicant: General Electric Company, Schenectady, NY (US)

(72) Inventors: Anthony John Murray, Lebanon, NJ (US); Andrew David Pris, Altamont, NY (US); Erik Leeming Kvam, Niskayuna, NY (US); Nandini Nagraj, Clifton Park, NY (US); Eugene Pauling Boden, Scotia, NY (US); Ernest William Kovacs, Cohoes, NY (US); Louisa Ruth Carr, Schenectady, NY (US); Rui Chen, Clifton Park, NY (US); Tiberiu Mircea Siclovan, Rexford, NY (US); Kelly Scott Chichak, Clifton Park, NY (US)

(21) Appl. No.: 15/071,210

(22) Filed: Mar. 16, 2016

(51) Int. Cl. C07K 1/36; C07K 16/00; C12N 9/74; C12N 15/115; C07K 14/705

(52) U.S. Cl. CPC C07K 1/36; C12N 15/115; C07K 14/70564; C12N 9/6429; C12Y 304/21005; C07K 16/00; C12N 2310/16; C12N 2310/351

(57) ABSTRACT

Provided herein are aptamer-polymer conjugates which are responsive to environmental stimuli and are useful in selective purification of untagged target polypeptides.

### STATEMENT REGARDING FEDERALLY SPONSORED RESEARCH & DEVELOPMENT

This invention was made with Government support under contract number HDTRA1-13-C-0096 awarded by US Government Defense Threat Reduction Agency (DTRA). The Government has certain rights in the invention.

### BACKGROUND

The disclosure relates generally to improved methods for separating and purifying biomolecules, in particular untagged polypeptides.

Large scale purification of biomolecules presents sui generis challenges. A number of currently approved biopharmaceuticals are polypeptides (e.g., antibodies). Biopharmaceutical synthesis in cell based production reactors is typically followed by downstream processing to remove contaminants that are unwanted in the formulated biopharmaceutical. Contaminants include host cell proteins, host cell DNA, endotoxins (in the case of bacterial production systems), viruses (in the case of mammalian production systems), misfolded proteins and aggregates, and components that leach from chromatographic media. The best purification performance is usually obtained only when multiple orthogonal separation modes are combined sequentially.

Affinity chromatography requires binding elements or affinity ligands, such as antibodies, for high selectivity and affinity. However, the utility of antibody-based binding agents is generally limited due to their size, cost, and complex structure. Engineered protein binders have proved to be successful as affinity ligands because of their small size, stability, and ease of synthesis in microbial production systems, but these binders might be unsuitable for therapeutic applications because of their potential immunogenicity. Further, protein-based binders are generally more expensive to produce for single-use applications, and may not be suitable for repeated use after column washing and regeneration steps.

For instance, a typical downstream processing platform for monoclonal antibody (mAb) purification utilizes Protein A chromatography. Prior to Protein A chromatography, it is necessary to clarify the medium from the bioreactor, since cells and cell debris can clog chromatography columns and significantly reduce column life. Process steps used for clarification generally include centrifugation followed by depth filtration (e.g., by using diatomaceous earth). Protein A affinity chromatography has become a standard in the biopharmaceutical industry, despite its high cost, because of the resulting purity (>95%) and concentration effect, which reduces the scale of subsequent process steps. However, a drawback of the Protein A method is that the conditions for elution from the Protein A column, and the hold time at low pH for virus inactivation can give rise to mAb aggregation. Aggregates in the final formulation are undesirable, since mAb aggregates are potentially immunogenic. The remaining purification steps include cation exchange chromatography (CEX) to remove host cell proteins (HCP), any undesired proteolytic cleavage products of Protein A from the affinity column, and mAb aggregates which generally elute on the tail of the mAb monomer fractions during CEX. A final anion exchange flow-through step is used to remove remaining HCP and host cell DNA. Since Protein A chromatography is not only the most expensive step in the process, but also the least scalable and non-disposable element, this part of the downstream purification process for polypeptides is an expensive bottleneck during large scale production.

Polymer-mediated affinity precipitation has been used for separation of polypeptides. Typically, the target polypeptide is tagged (e.g., covalently attached) with a compound (e.g., a cofactor, an oligonucleotide, an epitope, and the like) which facilitates the affinity precipitation. For instance, Fong et al. (Bioconjugate Chem. 1999, 10, 720-725) describe a purification of a polypeptide tagged with a first oligonucleotide which binds to a stimulus-responsive polymer conjugated to a second oligonucleotide which is complementary to the first oligonucleotide which enables a sequence specific hybridization of oligonucleotides conjugated to the protein and polymer. However, a drawback of the Fong method is that the tagged polypeptide has to be subjected to further cleavage and purification steps to remove the tag that is essential for the initial purification.

U.S. Pat. No. 9,217,048 describes affinity precipitation of polypeptides by use of polymers that are responsive to a change in ionic concentrations or a change in pH. The polymers described in U.S. Pat. No. 9,217,048 are heteropolymers (copolymers). A drawback of the affinity precipitation methods described in U.S. Pat. No. 9,217,048 is that the separation is non-specific because the polymer lacks a target-specific recognition element for selective precipitation of a target polypeptide.

There is a need in the field for scalable, reusable and cost-effective processes for selective purification of biomolecules subsequent to their synthesis in cell based or non-cell based production platforms. There is a need in the field for methods that allow for single-use and continuous use of non-protein binding-elements to identify and recover protein targets efficiently under milder conditions, while maintaining the structural and functional integrity of the target molecule.

### BRIEF DESCRIPTION

Provided herein are improved methods for selective purification of untagged polypeptides comprising the use of aptamers as binding elements that are conjugated to polymers that undergo phase transitions under the influence of environmental stimuli. The aptamer-polymer-conjugate-based affinity chromatography methods provided herein offer reduced cost, better stability, high selectivity and specificity towards a target and are suitable for single-use and continuous use paradigms.

In one aspect, provided herein is a method for selective purification of one or more than one untagged target polypeptide, the method comprising:
(a) contacting an aptamer-conjugated environmentally-responsive homopolymer with a mixture comprising one or more than one untagged target polypeptide; and
(b) precipitating the aptamer-conjugated environmentally-responsive homopolymer complexed to one or more than one untagged target polypeptide from the mixture by changing at least one property of the mixture;
wherein the environmentally-responsive homopolymer has a number average molecular weight greater than 11.5 kDa;
wherein steps (a) and (b) are carried out in the absence of a solid support or insoluble carrier material; and
wherein steps (a) and (b) are carried out under substantially matching pH conditions and salt concentrations.

Also provided herein are methods for reusing said aptamer-conjugated environmentally-responsive homopolymers in purification processes for untagged polypeptides.

### DRAWINGS

These and other features, aspects, and advantages of the present invention will become better understood when the following detailed description is read with reference to the accompanying drawings in which like characters represent like parts throughout the drawings, wherein:

FIG. 1A and FIG. 1B show a comparison of GPC data and NMR data for three polymers having number average molecular weights (Mn) of about 6 kDa, about 18 kDa and about 26 kDa and also shows polydispersity data for the three polymers synthesized according to Example 1.

FIG. 2A through FIG. 2F show sequential thermoprecipitation of three polymers having number average molecular weights (Mn) of about 6 kDa, about 18 kDa and about 28 kDa as described in Example 2. After the first thermoprecipitation, shown in FIG. 2A, the supernatant S1 was removed as shown in FIG. 2B and the thermoprecipitant was redissolved in a fresh 1 mL aliquot of HEPES buffer as shown in FIG. 2C, thermoprecipitated again to yield supernatant S2 as shown in FIG. 2D. A similar cycle provided supernatant S3 as shown in FIG. 2E and FIG. 2F. FIG. 2G shows UV-VIS absorbances for supernatant S1 of FIG. 2B. FIG. 2H shows UV-VIS absorbances for supernatant S2 of FIG. 2D. FIG. 2I shows UV-VIS absorbances for supernatant S3 of FIG. 2F. The final pellet was also redissolved in HEPES buffer and FIG. 2J shows UV-Vis analysis of the re-dissolved pellet after three rounds of thermoprecipitation.

FIG. 3 shows a synthetic scheme for synthesis of aptamer-conjugated environmentally-responsive homopolymers described herein. Example 3 provides details for the synthesis.

FIG. 4 shows a representative gel analysis of a pNIPAAM-aptamer conjugate of Example 3 and its associated supernatants (S1 and S2) obtained from two rounds of thermoprecipitation purification.

FIG. 5 provides a process map overview of selective protein thermoprecipitation and the details of the process are provided in Example 5.

FIG. 6A and FIG. 6B show the results of the thermoprecipitation experiments outlined in TABLES 2 and 3 and in Example 5, as gel analysis (FIG. 6A) and percent capture of L-selectin protein respectively (FIG. 6B).

FIG. 7A and FIG. 7B show the results of the thermoprecipitation experiments outlined in TABLES 2 and 3 and in Example 5, as gel analysis (FIG. 7A) and percent capture of thrombin protein (FIG. 7B) respectively.

FIG. 8A and FIG. 8B depict the results of the thermoprecipitation experiments outlined in TABLE 3 and in Example 6, as gel analysis (FIG. 8A) and percent capture of IgE antibody (FIG. 8B) respectively.

FIG. 9A and FIG. 9B depict the results of the thermoprecipitation experiments outlined in Example 7, as gel analysis of supernatants S1, S2, D1, D2, D3, (FIG. 9A) and as recovery from spent media (FIG. 9B) respectively.

FIG. 10A and FIG. 10B depict the results of the thermoprecipitation experiments outlined in Example 7, as gel analysis of supernatants S1, S2, D1, D2, (FIG. 10A) and as recovery from spent media (FIG. 10B) respectively.

FIG. 11A and FIG. 11B depict the results of thermoprecipitation experiments wherein the polymer-aptamer conjugate from the first round of thermoprecipitation (series A) was re-used for a second round of thermoprecipitation as outlined in Example 8. Data shown for gel analysis in FIG. 11A, where (1) is the gel analysis for the polymer-aptamer conjugate from the first round of thermoprecipitation and (2) is the gel analysis for the polymer-aptamer conjugate from the second round of thermoprecipitation; and data shown for percent capture of IgE antibody in FIG. 11B.

### DETAILED DESCRIPTION

Prior attempts at improving methods for purification of biomolecules, such as Fong et al. (Bioconjugate Chem. 1999, 10, 720-725), have involved the use of tagged polypeptides to achieve selective purification. However, such methods require additional steps for removal of the tag and subsequent purification. Alternatively, polymers which are responsive to changes in pH or ionic concentrations have been used for purification of untagged polypeptides such as methods described in U.S. Pat. No. 9,217,048. However such methods are non-selective. Moreover, the methods described in U.S. Pat. No. 9,217,048 utilize heteropolymers which contain charged groups that render the polymer responsive to changes in pH or ionic concentrations.

By contrast, the methods described herein allow for selective purification of polypeptides and also do not require that the polypeptides comprise tags as defined herein. In addition, the present methods utilize environmentally-responsive homopolymers as opposed to heteropolymers.

U.S. Patent Application Publication No. 20150093820 discloses conjugation of an oligonucleotide to a low molecular weight stimulus-responsive polymer having a number average molecular weight of ˜2000 g/mol. U.S. Pat. No. 6,258,275 describes affinity macroligands comprising certain stimulus-responsive polymers for precipitation of untagged avidin and discloses a preference for homopolymers of low number average molar mass (Mn) as a means for increasing the ligand density in the polymer.

Surprisingly, it was found that previously-described low molecular weight polymers (with or without conjugation to a ligand) did not precipitate efficiently from mixtures. It was found that efficient precipitation of the ligand-conjugated polymers is achieved only when the polymer molecular weights exceeds a threshold weight. Accordingly, the methods described herein do not utilize lower molecular weight environmentally responsive polymers (e.g., thermo-responsive homopolymers); instead, the methods described herein utilize environmentally responsive polymers (e.g., thermo-responsive homopolymers) having molecular weights of greater than 11 kDa, greater than 11.5 kDa, greater than 12 kDa, greater than 15 kDa or greater than 18 kDa. In some embodiments the methods described herein utilize environmentally responsive polymers (e.g., thermo-responsive homopolymers) having molecular weights of at least 11.3 kDa, 11.5 kDa, at least 12 kDa, at least 15 kDa or at least 18 kDa.

Advantageously, the methods described herein utilize aptamers which are conjugated to the environmentally-responsive homopolymers for selective purification of untagged polypeptides. Heretofore, the oligonucleotide-conjugated polymers described in the art required a complementary oligonucleotide tag on the target polypeptide to enable complexation of the target polypeptide and the polymer via sequence specific oligonucleotide hybridization. In contrast to previously known methods, the aptamer-conjugated environmentally-responsive polymers provided herein have not been described in the art. Aptamers differ from oliognucleotides because aptamers fold into unique three-dimensional conformations thereby allowing them to bind selectively to polypeptides even if the polypeptides are not tagged. The conditions that govern this unique conformation may or may not be compatible with the conditions required for purification of one or more than one target polyeptide. Described herein are purification conditions that are compatible with aptamer conformations as well as the one or more than one target polyeptide.

In one aspect, provided herein are affinity macroligands that comprise aptamers conjugated to higher molecular weight environmentally responsive polymers (e.g., a thermo-responsive polymer having a number average molecular weight greater than 11 kDa, or 11.5 kDa or 12 kDa); such affinity macroligands are advantageous because the aptamer moieties function as binding elements as well as recognition elements for the target polypeptides thereby removing the need for tagging the target polypeptides. A further advantage of the methods described herein is that the number of steps in a downstream large scale purification process is reduced because there is no need for tagging and un-tagging a target polypeptide. Yet another advantage of the methods described herein is that the step of contacting an affinity macroligand comprising an aptamer conjugated to a high molecular weight environmentally responsive polymer with one or more than one untagged target polypeptide, and the step of precipitating the target-macroligand complex, can be carried out without any substantial change to the pH and ionic concentration of the mixture which simplifies the downstream large scale purification process and reduces production costs. In addition, the aptamer-conjugated environmentally-responsive homopolymers can be re-used for multiple cycles of production without significant loss in purification efficiencies. The methods described herein do not require the presence of solid supports or non-dissolvable carriers during the purification steps, i.e., the present methods are fluid-state chromatography methods. The use of fluid chromatography renders the present methods amenable to single use and multiple/continuous use paradigms and also reduces the cost of production.

To more clearly and concisely describe and point out the subject matter of the claimed invention, the following definitions are provided for specific terms, which are used in the following description and the appended claims. Throughout the specification, exemplification of specific terms should be considered as non-limiting examples.

The singular forms “a”, “an” and “the” include plural referents unless the context clearly dictates otherwise. Approximating language, as used herein throughout the specification and claims, may be applied to modify any quantitative representation that could permissibly vary without resulting in a change in the basic function to which it is related. Accordingly, a value modified by a term such as “about” is not to be limited to the precise value specified. In some instances, the approximating language may correspond to the precision of an instrument for measuring the value. Similarly, “free” may be used in combination with a term, and may include an insubstantial number, or trace amounts while still being considered free of the modified term. Where necessary, ranges have been supplied, and those ranges are inclusive of all sub-ranges there between.

As used herein, “untagged target polypeptide” refers to a free target polypeptide, i.e., a target polypeptide which has not been conjugated to any moiety for the purpose of enabling recognition and/or binding and/or separation of the target polypeptide. By way of explanation, in one illustrative example, a tagged polypeptide used for affinity precipitation may comprise an oligonucleotide tag which allows for sequence specific hybridization to a complementary oligonucleotide ligand conjugated to a polymer. The corresponding untagged target polypeptide would be devoid of the oligonucleotide tag. An “untagged target polypeptide” may comprise a protein, a post-translationally modified protein, a peptide, or a synthetic peptide. An untagged target polypeptide is the molecule of interest, which either needs to be separated and purified out from a mixture of molecules or needs to be quantified or characterized. Exemplary untagged target polypeptide include a peptide, hormone, antibody, enzyme, antigenic peptide, vaccine, drug-conjugate, glycoprotein, or combinations thereof.

As used herein, “environmentally-responsive” polymer is a polymer which is responsive to a change in one or more properties or parameters (e.g., a physical or chemical change in the environment), such as temperature, humidity, pH, conductivity, the wavelength or intensity of light, an electrical or magnetic field, ultrasonic wave, and the like, which results in a response. A “thermo-responsive” polymer is a polymer which undergoes a change in solubility in response to a change in temperature. By way of example, a thermo-responsive polymer may be soluble at a temperature below the LCST of the polymer but may precipitate out of the solution upon heating the solution to a temperature higher than the LCST of the polymer.

As used herein, “homopolymer” is a type of polymer synthetically derived from a single type of input monomer in which each monomer possesses an identical discreet molecular weight, elemental composition, isotopic composition, and (if relevant) isomeric form, tautomeric form, and/or chirality.

As used herein, a “number average molecular weight” of a polymer refers to the statistical average molecular weight of all the polymer chains in the sample and is calculated by dividing the total weight of all the polymer molecules in a sample by the total number of polymer molecules in a sample.

As used herein, in one embodiment, “substantially matching pH” means a pH difference of up to about 10% from the initial pH. In another embodiment, substantially matching pH means identical pH. In yet another embodiment, substantially matching pH means a pH difference of up to about 20% from the initial pH.

As used herein, in one embodiment, “substantially matching salt concentration” means a salt concentration difference of up to about 10% from the initial salt concentration. In another embodiment, substantially matching salt concentration means identical salt concentration. In yet another embodiment, substantially matching salt concentration means a salt concentration difference of up to about 20% from the initial salt concentration.

As used herein, “aptamers” are binding elements that efficiently bind to a target molecule through one or more binding sites through different types of conformational and physicochemical interactions. The aptamer may be a single stranded (DNA) aptamer, a single stranded ribonucleic acid (RNA) aptamer, a peptide nucleic acid (PNA) aptamer, or a combination of these types. Those skilled in the art will recognize that aptamers are functionally distinct from oligonucleotides and polypeptides. In some embodiments, the aptamer may comprise modified nucleotides that increase the folding diversity of the aptamer. In a further embodiment, the aptamer may comprise chemical modifications or side-chain fusions that protect the aptamer from enzymatic or chemical degradation. Aptamers may also comprise peptide bonds rather than phosphodiester bonds. In some embodiments, one or more than aptamer may be conjugated onto an environmentally-responsive polymer to bind one or more than one polypeptide. In some embodiments, an aptamer is a “randomer” and comprises a randomized aptamer sequence. In some embodiments an aptamer comprises locked nucleic acid (LNA) modifications.

As used herein “precipitate” includes solids, colloidal solids, emulsions, and/or any partitioned or collapsed material comprising aptamer-polymer conjugate or aptamer-polymer conjugate complexed with an untagged target polypeptide which can be separated using any technique including and not limited to filtration, electrostatic, ultrasonic, or magnetic means (e.g., in the presence of electrostatic, ultrasound, or magnetic field), centrifugation, decantation, sedimentation, gravity settling, and the like. As used herein “precipitating” or “precipitation” includes obtaining and/or causing the formation of a “precipitate”. In some embodiments, precipitating, or the act of precipitation, includes flocculation prior to removal from solution.

In one aspect, provided herein is a method for selective purification of one or more than one untagged target polypeptide, the method comprising:
(a) contacting an aptamer-conjugated environmentally-responsive polymer with a mixture comprising one or more than one untagged target polypeptide; and
(b) precipitating the aptamer-conjugated environmentally-responsive polymer complexed to one or more than one untagged target polypeptide from the mixture by changing at least one property of the mixture;
wherein the environmentally-responsive polymer has a number average molecular weight greater than 11 kDa;
wherein steps (a) and (b) are carried out in the absence of a solid support or insoluble carrier material; and
wherein steps (a) and (b) are carried out under substantially matching pH conditions and salt concentrations.

In some embodiments the environmentally-responsive polymer is a thermo-responsive polymer.

In another aspect, provided herein is a method for selective purification of one or more than one untagged target polypeptide, the method comprising:
(a) contacting an aptamer-conjugated environmentally-responsive homopolymer with a mixture comprising one or more than one untagged target polypeptide; and
(b) precipitating the aptamer-conjugated environmentally-responsive homopolymer complexed to one or more than one untagged target polypeptide from the mixture by changing at least one property of the mixture;
wherein the environmentally-responsive homopolymer has a number average molecular weight greater than 11.5 kDa;
wherein steps (a) and (b) are carried out in the absence of a solid support or insoluble carrier material; and
wherein steps (a) and (b) are carried out under substantially matching pH conditions and salt concentrations.

Typically, affinity chromatography purifications are carried out in the presence of solid supports or insoluble carrier materials which can be removed from the mixture during the purification process. By contrast, the methods provided herein do not require such solid supports or insoluble carrier materials because the environmentally-responsive aptamer-conjugated homopolymers (affinity macroligands) themselves function as supports/carriers during the purification process. In some embodiments, an excess of the environmentally-responsive homopolymer (or monomers thereof) is added to the reaction during the conjugation of the aptamer to the environmentally-responsive homopolymer whereby the environmentally-responsive aptamer-conjugated homopolymers and the non-conjugated environmentally-responsive homopolymers present in the mixture together function as carrier polymers (e.g., soluble carrier material) for the purification process. Accordingly, the selective purification methods described herein comprise affinity chromatography that is carried out in in the absence of a solid support or insoluble carrier material, or in the alternative, in the presence of soluble carrier material. In other words, the methods of selective purification of non-tagged polypeptides described herein comprise using affinity macroligands which comprise a binding/recognition element (e.g., aptamer) conjugated to the carrier material (e.g., environmentally-responsive polymer).

In some embodiments the environmentally-responsive homopolymer is a thermo-responsive homopolymer.

In some of the embodiments described above, changing at least one property of the mixture comprises changing the temperature of the mixture such that step (a) is carried out at a first temperature and step (b) is carried out at a second temperature which is different from the first temperature. Moreover, the steps (a) and (b) are carried out without changing the pH or salt concentration of the mixture, or at substantially the same pH and salt concentrations. The use of substantially the same pH and salt concentrations in steps (a) and (b) reduces or avoids changes to the conformation of the aptamer, thereby allowing for pulling down/capture of the one or more than one untagged polypeptide target with the aptamer-polymer conjugate. In some of such embodiments, where the environmentally-responsive homopolymer is a thermo-responsive homopolymer, the first temperature is below the lower critical solution temperature (LCST) of the aptamer-conjugated thermo-responsive homopolymer and the second temperature is higher than the lower critical solution temperature of the aptamer-conjugated thermo-responsive homopolymer. In some of such embodiments, the LCST of the aptamer-conjugated thermo-responsive homopolymer is less than about 45° C. In some other such embodiments, the LCST of the aptamer-conjugated thermo-responsive homopolymer is less than about 40° C.

In some embodiments, the polypeptide comprises a protein, an antigenic peptide, a vaccine, an enzyme, antibody, a drug-conjugate, a glycoprotein, or a combination thereof.

In some embodiments, the aptamer-conjugated environmentally-responsive homopolymer complexed to one or more than one untagged target polypeptide is precipitated when at least one property of the mixture of step (a) is changed. In such embodiments, the methods further comprise step (c): separating the precipitated aptamer-conjugated environmentally-responsive homopolymer complexed to one or more than one untagged target polypeptide from the mixture by filtration, gravitational settling, centrifugation, electrostatic means, ultrasonic means, or magnetic means.

In some embodiments of the methods described above, the methods further comprise step (d): treating the separated aptamer-conjugated environmentally-responsive homopolymer complexed to one or more than one untagged target polypeptide with a condition that induces dissociation of the aptamer-conjugated environmentally-responsive homopolymer from the one or more than one untagged target polypeptide (to provide a “stripped” aptamer-conjugated environmentally-responsive homopolymer and “free” one or more than one untagged target polypeptide); step (e): precipitating the dissociated (“stripped”) aptamer-conjugated environmentally-responsive homopolymer by changing at least one property of the mixture of step (d); and step (f): isolating a selectively-purified untagged polypeptide from the supernatant of step (e).

In one embodiment, the aptamer-conjugated environmentally-responsive homopolymer complexed to one or more than one untagged target polypeptide is precipitated by heating the mixture of step (a) to a temperature above LCST of the environmentally-responsive hompolymer. In some embodiments, the precipitation of the stripped aptamer-conjugated environmentally-responsive homopolymer is achieved by heating the mixture of step (d) to a temperature above LCST of the environmentally-responsive hompolymer.

In some embodiments of the methods described above, the methods further comprise step (g): reusing the precipitated aptamer-conjugated environmentally-responsive homopolymer of step (e) for selective purification of one or more untagged polypeptide in a continuous manner, wherein the precipitated aptamer-conjugated environmentally-responsive homopolymer is resuspended with a new mixture comprising one or more than one untagged target polypeptide.

In some embodiments of the methods described above, the condition that induces dissociation of the aptamer from the one or more than one untagged target polypeptide is selected from the group consisting of suspension in water, applying a chelating agent, a pH which is different from the pH of the mixture in step (a), a temperature which is different from the temperature of the mixture in than step (a) and step (b); a salt concentration which is different from the salt concentration of the mixture in step (a), or a combination thereof. In such embodiments, the dissociation conditions lead to formation of a “stripped” aptamer-conjugated environmentally-responsive homopolymer and “free” one or more than one untagged target polypeptide.

In some embodiments, the homopolymer is poly(N-isopropylacrylamide) (pNIPAM). In further embodiments, the homopolymer is poly(N,N-diethylacrylamide) (PDEAAm). In some embodiments, the homopolyer is poly(N-vinlycaprolactam) (PVCL). In yet further embodiments, the homopolymer is poly[2-(dimethylamino)ethyl methacrylate] (PDMAEMA). In some alternative embodiments, the homopolymer comprises comprises poly(ethylene glycol) methacrylate (PEGMA) or poly(ethylene glycol) (PEG), alternatively known as poly(ethylene oxide) (PEO). The homopolymer can be linear or comprise pendant or brush-like chains.

In one group of embodiments, the environmentally-responsive homopolymer has a number average molecular weight greater than 12 kDa. In another group of embodiments, the environmentally-responsive homopolymer has a number average molecular weight greater than 15 kDa.

In some embodiments of steps (a) to (g) described above, the environmentally-responsive homopolymer is a thermo-responsive homopolymer.

In one aspect, described herein is a method for selective purification of one or more than one untagged target polypeptide, the method comprising:
(a) contacting an aptamer-conjugated thermo-responsive homopolymer with a mixture comprising one or more than one untagged target polypeptide; and
(b) precipitating the one or more than one untagged target polypeptide complexed with the aptamer-conjugated thermo-responsive homopolymer from the mixture by changing the temperature of the mixture;
wherein the thermo-responsive homopolymer has a number average molecular weight greater than 11.5 kDa;
wherein steps (a) and (b) are carried out in the absence of a solid support or insoluble carrier material; and
wherein steps (a) and (b) are carried out under substantially matching pH conditions and salt concentrations.

In a further aspect, provided herein is a method for selective purification of one or more than one untagged target polypeptide, the method comprising:
(a) contacting an aptamer-conjugated thermo-responsive homopolymer with a mixture comprising one or more than one untagged target polypeptide; and
(b) precipitating the one or more than one untagged target polypeptide complexed with the aptamer-conjugated thermo-responsive homopolymer from the mixture by changing the temperature of the mixture;
wherein the LCST of the aptamer-conjugated thermo-responsive homopolymer is less than about 45° C.;
wherein steps (a) and (b) are carried out in the absence of a solid support or insoluble carrier material; and
wherein steps (a) and (b) are carried out under substantially matching pH conditions and salt concentrations.

In some of such embodiments, the thermo-responsive homopolymer has a number average molecular weight greater than 11.5 kDa. In some of such embodiments, the thermo-responsive homopolymer has a number average molecular weight greater than 12 kDa. In some other such embodiments, the thermo-responsive homopolymer has a number average molecular weight greater than 15 kDa. All the features described above for methods comprising the use of aptamer-conjugated environmentally-responsive homopolymers are applicable to methods comprising the use of aptamer-conjugated thermo-responsive homopolymers and are expressly contemplated herein.

In a further aspect, provided herein is a method for selective purification of one or more than one untagged target polypeptide, the method comprising:
(a) contacting an aptamer-conjugated pNIPAM homopolymer with a mixture comprising one or more than one untagged target polypeptide;
(b) precipitating the one or more than one untagged target polypeptide complexed to the aptamer-conjugated pNIPAM homopolymer from the mixture by changing the temperature of the mixture;
(c) separating the precipitated aptamer-conjugated pNIPAM homopolymer complexed to the one or more than one untagged target polypeptide from the supernatant by filtration or gravitational settling or centrifugation;
(d) treating the separated aptamer-conjugated pNIPAM homopolymer complexed to the one or more than one untagged target polypeptide with a condition that induces dissociation of the aptamer-conjugated pNIPAM homopolymer from the one or more than one untagged target polypeptide;
(e) precipitating the dissociated aptamer-conjugated pNIPAM homopolymer by changing the temperature of the mixture of step (d);
(f) isolating the one or more than one untagged polypeptide from the supernatant of step (e); and
(g) optionally reusing the precipitated aptamer-conjugated pNIPAM homopolymer from step (e) for repeating steps (a) to (f);
wherein the pNIPAM homopolymer has a number average molecular weight greater than 11.5 kDa;
wherein steps (a) and (b) are carried out in the absence of a solid support or insoluble carrier material; and
wherein steps (a) and (b) are carried out under substantially matching pH conditions and salt concentrations.

In some embodiments, the condition that induces dissociation of the aptamer from the one or more than one untagged target polypeptide is selected from the group consisting of suspension in water, applying a chelating agent, a pH which is different from the pH of the mixture in step (a), a temperature which is different from the temperature of the mixture in step (a) and step (b); a salt concentration which is different from the salt concentration of the mixture in step (a), or a combination thereof. Non-limiting exemplary conditions for dissociation include 0.5M-1M salt solutions, 5-50 mM EDTA, or 10 mM glycine-HCl within an acidic range of about pH2 to about pH6. In some embodiments, changes in pH above or below the pI of the target polypeptide are suitable for disassociation of the aptamer from the polypeptide. In some embodiments, water is sufficient for dissociation of the aptamer from the polypeptide. In other embodiments, where aptamers include modified bases to improve hydrophobic interactions with the target polypeptide, solvent manipulation may be required for dissociation. In another exemplary embodiment, reducing or avoiding the presence of potassium in a binding buffer led to improved conditions for dissociation of the aptamer from the polypeptide.

In some embodiments, the aptamer-conjugated pNIPAM homopolymer has an LCST of less than about 45° C. In some embodiments, the LCST of the aptamer-conjugated pNIPAM homopolymer is less than about 40° C. In some embodiments, the LCST of the aptamer-conjugated pNIPAM homopolymer is less than about 35° C.

In one group of embodiments, the pNIPAM homopolymer has a number average molecular weight greater than 12 kDa. In other embodiments, the pNIPAM homopolymer has a number average molecular weight greater than 15 kDa.

All the features described above for methods comprising the use of aptamer-conjugated environmentally responsive homopolymers (e.g., aptamer-conjugated thermo-responsive homopolymers) are applicable to methods comprising the use of aptamer-conjugated pNIPAM homopolymers and are expressly contemplated herein.

In one embodiment, a thermo-responsive polymer may include poly(N-vinyl caprolactam), poly(N,N-diethylacrylamide), poly(N-isopropylacrylamide), poly[2-(dimethylamino)ethyl methacrylate], poly(ethylene glycol) methacrylate (PEGMA) or poly(ethylene glycol) (PEG), alternatively known as poly(ethylene oxide) (PEO), or combinations thereof. While several thermoresponsive polymers have been investigated, the LCST of 32° C. for pNIPAM makes it preferable for use in biological systems. The LCST of 32° C. is in a range compatible with the thermostability of the majority of biomolecules. However, alternative thermo-responsive polymers and combinations thereof are also contemplated within the scope of embodiments provided herein.

In some embodiments, the aptamer-conjugated thermo-responsive polymer has an LCST less than about 45° C. In some embodiments, the aptamer-conjugated thermo-responsive polymer has an LCST less than about 40° C. In some embodiments, the aptamer-conjugated thermo-responsive polymer has an LCST less than about 35° C. In some embodiments, the aptamer-conjugated thermo-responsive polymer has an LCST between about 30° C. and about 45° C. In some embodiments, the aptamer-conjugated thermo-responsive polymer has an LCST between about 30° C. and about 40° C. One of skill in the art will recognize that the LCST of thermo-responsive polymers can be directly-dependent, inversely-dependent, or independent of the end-groups attached onto the polymer, with the magnitude of change depending on the chemical nature of the end-group. One of skill in the art will recognize that the LCST of thermo-responsive polymers can increase in a manner dependent on molecular weight when hydrophilic end groups are attached to the polymer. Further, one of skill in the art will recognize that the LCST of a thermo-responsive polymer, an aptamer-conjugated thermo-responsive polymer, and/or an aptamer-conjugated thermo-responsive polymer complexed with one or more than one untagged polypeptides may be substantially similar and any minor variations fall within the spirit and scope of the methods described herein.

The present methods may suitably comprise, consist of, or consist essentially of one or more than one of any of the following: aptamers conjugated to environmentally-responsive polymers, aptamers conjugated to environmentally-responsive homopolymers, aptamers conjugated to thermo-responsive polymers, aptamers conjugated to thermo-responsive homopolymers, substantially matching pH conditions, substantially matching salt concentrations, environmentally-responsive polymers having a number average molecular weight greater than 11 kDa, or greater than 11.5 kDa, or greater than 12 kDa, environmentally-responsive homopolymers having a number average molecular weight greater than 11 kDa, or greater than 11.5 kDa, or greater than 12 kDa, thermo-responsive polymers having a number average molecular weight greater than 11 kDa, or greater than 11.5 kDa, or greater than 12 kDa, thermo-responsive homopolymers having a number average molecular weight greater than 11 kDa, or greater than 11.5 kDa, or greater than 12 kDa, or are carried out in the absence of a solid support or insoluble carrier material, or in the presence of a polymeric carrier, or in the presence of a soluble carrier, or any combination thereof.

### EXAMPLES

### Example 1: Synthesis and Characterization of Amine-Terminated pNIPAM Homopolymers at Defined Molecular Weights

### Polymerization of poly(N-isopropylacrylamide (pNIPAM)

In the examples described herein Me6TREN (hexamethyltriethylenetetramine, Tris[2-(dimethylamino)ethyl]amine) was the ligand of choice and was used without further purification; CuBr was purified by degassed water precipitation from HBr and then dried and stored under nitrogen. N-isopropylacrylamide monomer was used without further purification. Functionalized initiators were purified by normal phase MPLC (ISCO, silica Gold columns). Deionized water and reagent grade dimethylformamide (DMF) were degassed and stored under nitrogen gas.

Unless otherwise stated, the ratio of initiator to Me6TREN to CuBr was 1:0.6:0.8. The ratio of monomer to initiator was varied according to the desired Mn of the polymer (i.e. low, medium, or high Mn), and the reaction volume was scaled accordingly.

To begin a typical procedure (here, a target Mn 15,800 polymer is used as an example), a polymerization flask containing CuBr (36 mg, 0.25 mmol) and a football stir bar was capped with a rubber septum and purged with nitrogen gas for 20 min During this time, a pear-shaped flask containing Me6TREN (33 mg, 0.19 mmol) and water (13 ml) was degassed by bubbling nitrogen gas for 20 minutes using a transfer canula for the gas outlet. A second pear-shaped flask was used to weigh the monomer (5 g, 44.18 mmol) and initiator. If the initiator was not completely soluble in water, it was added as a solution in up to 1.6 ml DMF. Water (24 ml less the DMF volume) was added and the mixture was degassed similarly to the ligand solution (20 min).

The ligand solution was transferred via canula, under nitrogen gas, into the polymerization flask immersed in an ice bath, and the mixture was stirred at 0° C. for 20 minutes, during which time a Cu(I) disproportionation occurred: the solution turned blue from Cu(II) in the presence of flocculated Cu(0). At this point, the monomer-initiator solution was added via canula, still under nitrogen gas, and the polymerization was conducted at 0° C. for at least 4 hrs, preferably overnight.

At the end of the reaction, the reaction solution was heated to 40° C. to precipitate the polymer. The aqueous layer (supernatant) was decanted. The precipitated polymer fraction was cooled by adding 20 ml water, and precipitating again. The polymer was dried by repeatedly dissolving and concentrating it from methanol (50 ml, 2×) and then chloroform (50 ml, 2×). The clear concentrated chloroform solution (˜50 ml) was then passed through a plug (¾ in) of basic alumina, the adsorbent rinsed twice with ˜75 ml chloroform, concentrated under reduced pressure to about 10 ml, precipitated in diethyl ether (Et₂O) (800 ml) and dried in vacuum overnight.

Characterization of pNIPAM Molecular Weight and Dispersity

Molecular weight determination was performed by gel permeation chromatography using polystyrene standards for calibration and conducted with an Agilent 1100 equipped with a refractive index detector using a PLgel 5 μm MIXED-C Agilent column (300×7.5 mm) with 1.0% isopropanol in chloroform as an eluent at 35° C., at a flow rate of 1 mL/min and at a concentration of 1.0 mg/mL. The samples were prepared using the same mobile phase with 0.5% anisole added as an internal standard.

Molecular weight was also evaluated using end-group analysis performed on solutions of ˜0.1 mg polymers dissolved in 1 ml deuterated water. NMR spectra were collected on a Bruker Avance, 400 MHz Nuclear magnetic resonance instrument. 64 scans per spectrum were used to maximize signal-to-noise for reliable end-group analysis. The relative integration of the peaks from the initiator to the integration of the isopropyl proton on the polymer (1 proton per repeat unit within each polymer) yields a ratio of end-groups to repeat units. In other words, the average degree of polymerization can be determined, and thus the average molecular weight of the polymer population can be calculated.

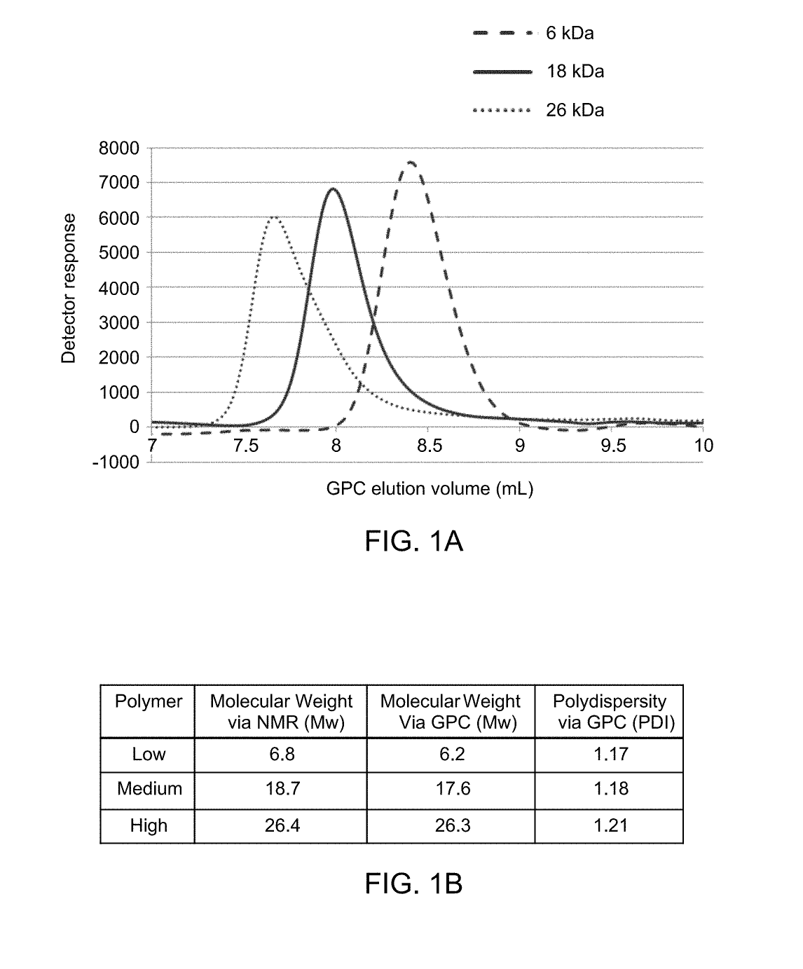

FIG. 1A and FIG. 1B summarize the characterization of three polymers with distinct molecular weight distributions. GPC analysis of the “Low” molecular weight polymer revealed an Mn around 6.2 kDa with a polydispersity index (PDI) of 1.17, which NMR end-group analysis confirmed as Mw around 6.8 kDa. The molecular weight of “Medium” polymer was found to be 18.7 kDa by NMR end-group analysis and 17.6 kDa by GPC, with a PDI of 1.18. “High” molecular weight polymer was found to have 26.4 kDa size by NMR and 26.3 kDa by GPC, with a PDI of 1.21. While there is some overlap of the traces in GPC, the bulk properties of the populations (high, medium, or low) will be representative of the majority of the polymer chains in each distribution. Multiple batches of pNIPAM were produced under conditions similar to the conditions described above and yielded similar PDI ranges, although the resulting molecular weights exhibited some expected differences based on slight variations in starting conditions and inputs.

### Example 2: The Molecular-Weight Dependency of Polymer Thermoprecipitation

Small aliquots (20-100 mg) of amine-terminated pNIPAM polymer prepared according to the procedure in Example 1 were reacted with excess Alexa Fluor 647 (AF647) NHS ester dye (Thermo Fisher, A-20006) to yield fluorescently-labeled polymer conjugates. Dye-labeling reactions were carried out under identical conditions for each of three different (6 kDa, 18 kDa, and 28 kDa) pNIPAM molecular weight distributions. The reactions proceeded as follows: AF647 NHS ester was dissolved in anhydrous DMF and immediately added to freshly prepared 3 μM pNIPAM solutions in buffer (10 mM HEPES, pH 7.3), such that 10-fold molar excess of pNIPAM was used. Total reaction volumes were 1 mL. After thorough mixing, the reactions were protected from light and incubated at ambient temperature for 30 min before a further overnight incubation at 4° C.

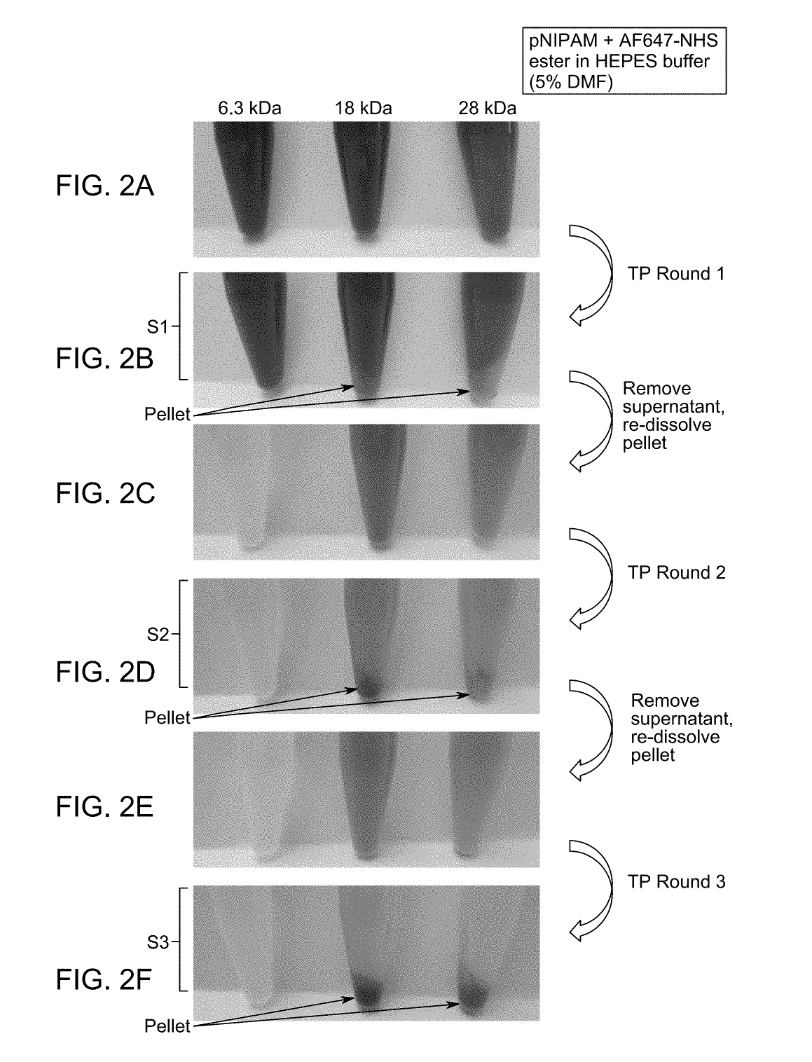

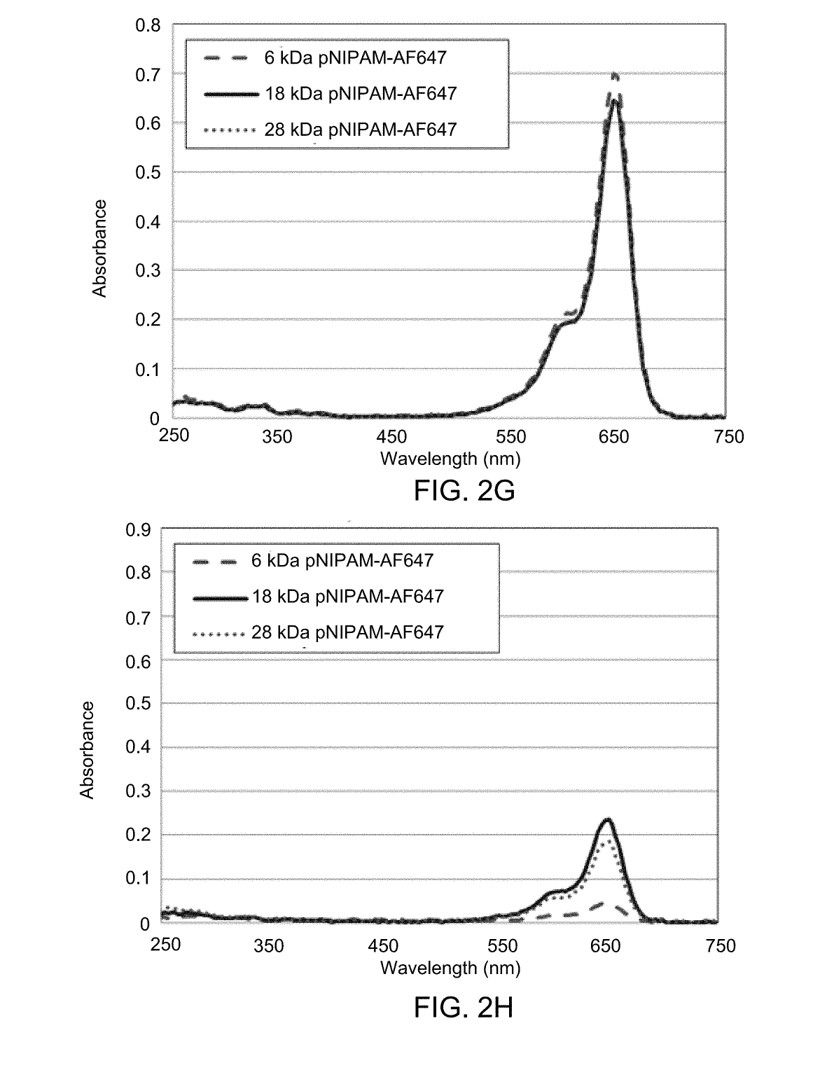

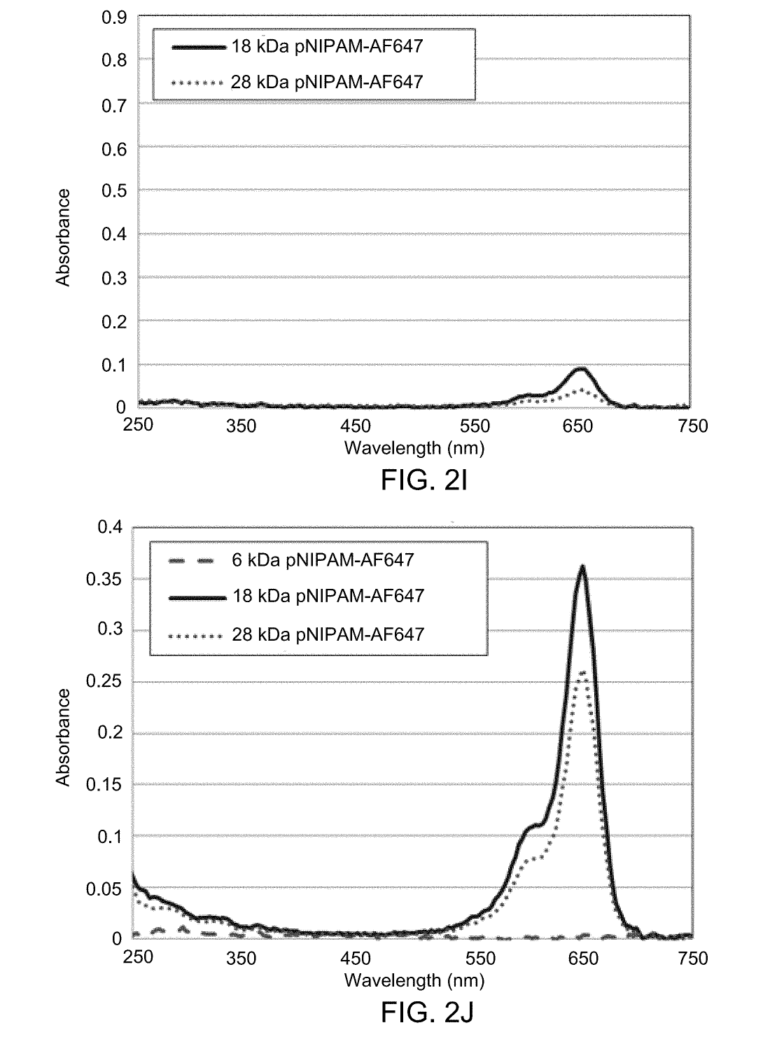

FIG. 2A (top picture) depicts identical blue-tinted crude reaction mixtures after overnight incubation. Following an initial round of thermoprecipitation (TP) by centrifuging at elevated temperature (30 min, 40° C., 12,000×g ref), a bluish-white pellet was clearly observed in the 18 k and 28 k samples, while no precipitate was visible for the 6 k sample (FIG. 2B). Supernatant removal (S1) followed by re-dissolving each thermoprecipitant in a fresh 1 mL aliquot of HEPES buffer yielded the expected blue solution mixtures for 18 k and 28 k with a nearly colorless appearance for 6 k (FIG. 2C). The first round of thermoprecipitation (TP) underscores the inability of the lower molecular weight pNIPAM to efficiently precipitate or pellet under physiological ionic conditions. Two additional rounds of TP (generating supernatants S2 and S3) (FIG. 2D, FIG. 2E, FIG. 2F) further accentuated these differences while highlighting the ability of the higher molecular weight pNIPAM to undergo repeated cycles of TP with the expected depletion of only the unreacted or “free” AF647 dye. While a cloudy appearance was observed for the 6 kDa sample (with or without fluorescent dye attachment) upon heating at 40° C. for >5 min, no pellet was obtained under the tested TP conditions. It was observed that the polymers with Mn of 18 kDa and 28 kDa provided pellets upon heating at 40° C. for >5 min (i.e., had LCSTs<40° C.) while polymers with lower Mn (6 kDa) did not provide pellets upon heating at 40° C. for >5 min (i.e., had LCSTs>40° C.). Several literature examples (Chung, J. E. et al. J. Controlled Release 1998, 53, 119-130; Xia, Y. et al. Macromolecules 2005, 38, 5937-5943; XingPing Q. et al. Sci China Chem 2013, 56, 56-64) also provide evidence of higher LCSTs (LCST>40° C.) for low molecular weight pNIPAM functionalized with a terminal polar or hydrophilic group either as the initiator or upon subsequent modification of the initiator.

FIG. 2G depicts UV-VIS absorbance readings for supernatant S1 of FIG. 2B. FIG. 2H shows UV-VIS absorbances for supernatant S2 of FIG. 2D. FIG. 2I shows UV-VIS absorbances for supernatant S3 of FIG. 2F. The final pellet was also redissolved in HEPES buffer and FIG. 2J shows UV-Vis analysis of the re-dissolved pellet after three rounds of thermoprecipitation.

These readings were obtained via NanoDrop measurements performed in triplicate. The UV-Vis analysis of the re-dissolved pellets after three rounds of TP depicts a slightly higher dye absorbance signal for 18 k relative to 28 k. This indicates a higher degree of dye labeling obtained for the shorter 18 k polymer which may be presumed to possess a more accessible terminal reactive amine relative to the longer 28 k. Based on the UV-Vis absorbance measurements, the AF647 labeling efficiency for 18 k is calculated to be 5.0%, while the efficiency is 3.7% for the 28 k polymer.

### Example 3: Synthesis of pNIPAM-Aptamer Conjugates

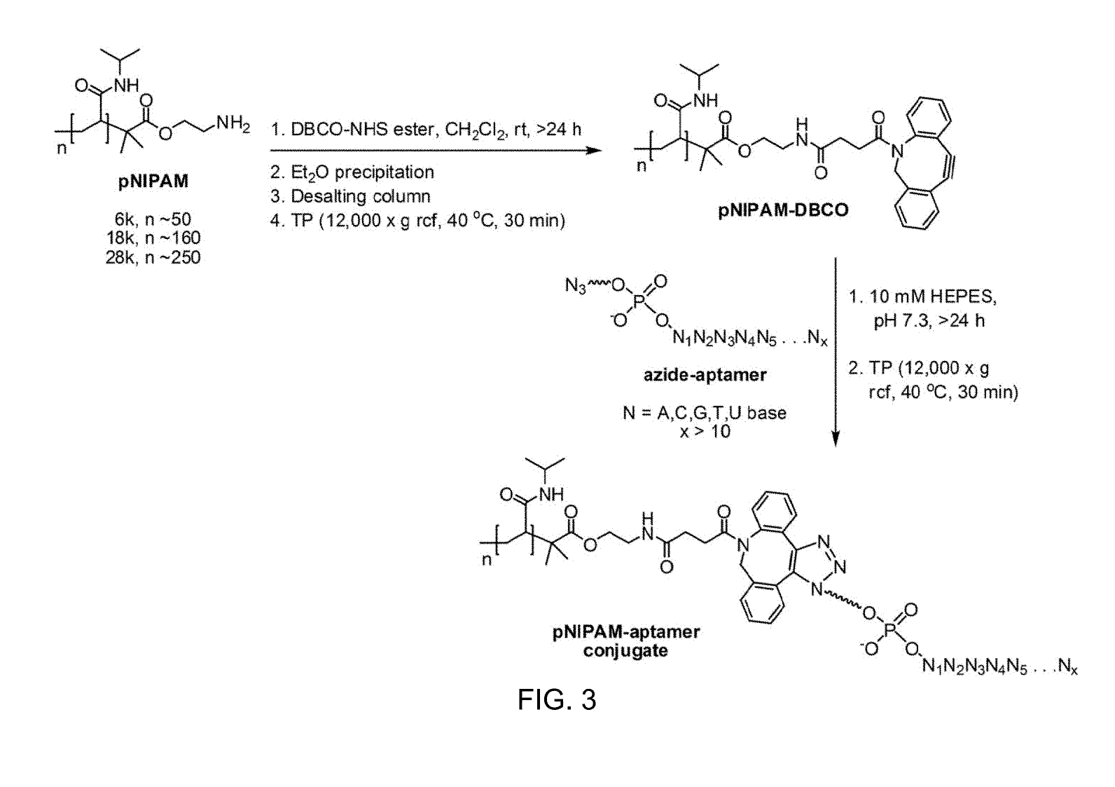

The synthetic preparation of pNIPAM-aptamer conjugates for analyte purification proceeded according to the general protocol described below (and outlined in FIG. 3) for all aptamers and all pNIPAM polymers with an approximate average molecular weight >11 kDa.

Synthesis of pNIPAM-DBCO Intermediate

Previously synthesized, purified, and characterized amine-terminated pNIPAM polymer (200-300 mg, 12 umol) was added with excess (>10 molar equivalents) of DBCO-NHS ester (Click Chemistry Tools, A133) to 4-6 mL of anhydrous methylene chloride. The resulting reaction mixture was vigorously stirred at room temperature for >24 h under anhydrous conditions. Following partial reduction of reaction volume, diethyl ether was added to the mixture at ˜5-fold (v/v) excess to allow for polymer precipitation as a white solid. After several minutes of mixing, the sample was centrifuged at low speed (500×g ref, 1 min) followed by supernatant removal. After re-dissolving the precipitate in a minimum volume of methylene chloride, the polymer was again precipitated by and washed with ether an additional two times.

After drying the resulting solid under reduced pressure, ˜50 mg aliquots of samples were re-dissolved in pure water (˜1 mL) and applied to 5 mL Zeba desalting columns for additional removal of truncated polymer, unreacted initiator, and other small molecule impurities (7K MWCO columns, Thermo Fisher). Recovered fractions of polymer were then additionally subjected to two rounds of TP using our standard conditions for centrifugation at elevated temperature (30 min, 40° C., 12,000×g ref). DBCO labeling efficiency was measured using the characteristic DBCO absorbance peak at 309 nm (molar extinction coefficient=12,000 M⁻¹ cm⁻¹). Thus, known quantities of total polymer in solution were compared to DBCO concentration as determined by NanoDrop UV-Vis readings. Typical final, isolated yields of pNIPAM-DBCO were 10-40% depending on the average polymer length and number of initial input DBCO-NHS ester equivalents used.

Conjugation of Aptamer to pNIPAM-DBCO

Purified pNIPAM-DBCO was re-dissolved in HEPES buffer (10 mM, pH 7.3) at ˜20 μM DBCO before addition to azide-aptamer aliquots derived from 1 mM HEPES stock solutions. Azide-aptamer was typically present in 3-5-fold molar excess relative to polymer-bound DBCO content. The resulting copper-free “Click” reactions were allowed to proceed for >24 h at room temperature. Purification of the resulting pNIPAM-aptamer conjugates was achieved via three rounds of TP using standard conditions. Aptamer attachment efficiency was assessed via NanoDrop UV-Vis readings along with analysis of denaturing 15% TBE-Urea DNA gels run at 170V for 90 min on ice and followed by SYBR Gold staining and Typhoon (GE Healthcare) fluorescence imaging. Final isolated yields for pNIPAM-aptamer conjugates ranged from 1-10% depending on the polymer length and the aptamer used.

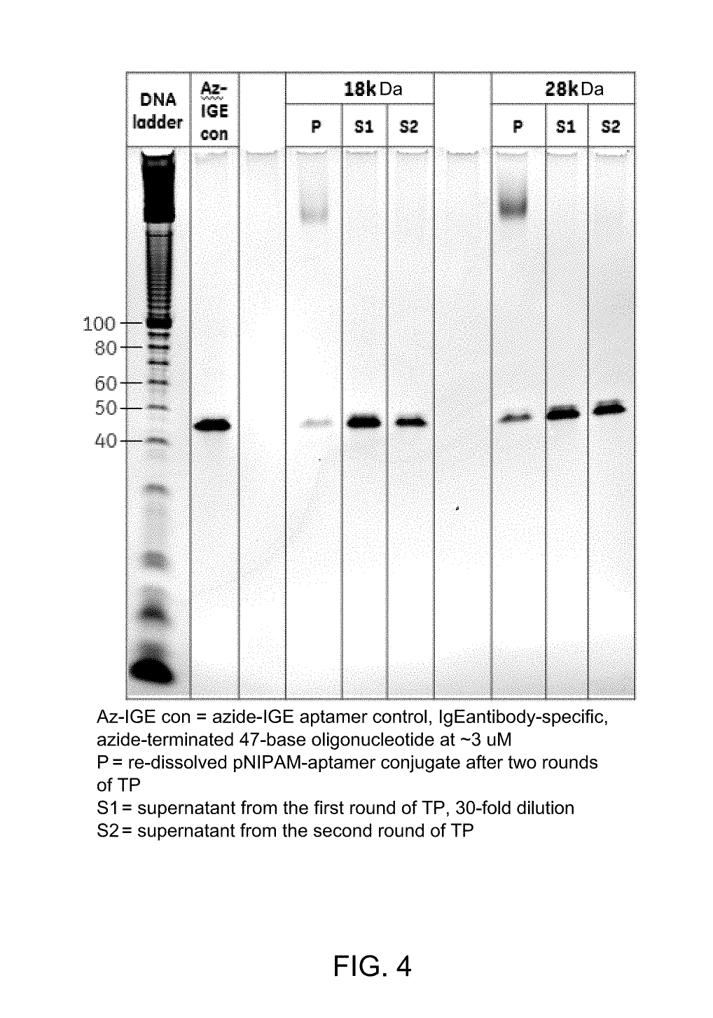

FIG. 4 depicts a representative gel analysis of a pNIPAM-aptamer conjugate and its associated supernatants (S1 and S2) obtained from two rounds of TP purification. For this particular example, the starting aptamer, azide-IGE (Az-IGE, SEQ ID NO: 4), is an IgE antibody-specific, azide-terminated 47-base oligonucleotide. The high molecular weight bands in the P-labeled lane indicate pNIPAM-aptamer conjugate derived from re-dissolved pellet samples following two rounds of TP, while the 47b low molecular weight bands depict Az-IGE starting material.

Other pNIPAM-aptamer conjugates are synthesized using similar synthetic protocols for which certain aptamers and randomers are provided in TABLE 1 below.

TABLE 1

| Aptamer | SEQ ID | | |
|---|---|---|---|
| ID | NO | Sequence (N-term-C-term; 5′→3′) | Length |
| L- | SEQ ID | /5AzideN/TTTTTTTTTTTAGCCAAGGTAA | 59 |
| Selectin | NO: 1 | CCAGTACAAGGTGCTAAACGTAATGGC | |
| aptamer | | TTCGGCTTAC | |
| Thrombin | SEQ ID | /5AzideN/TTTTTTTTTTGGTTGGTGTGGT | 25 |
| aptamer | NO: 2 | TGG | |
| Thrombin | SEQ ID | /5AzideN/TTTTTTTTTTGGTGGTGGTTGT | 25 |
| randomer | NO: 3 | GGT | |
| IgE | SEQ ID | /5AzideN/TTTTTTTTTTGGGGCACGTTTA | 47 |
| aptamer | NO: 4 | TCCGTCCCTCCTAGTGGCGTGCCCC | |
| Extended | SEQ ID | /5AzideN/TTTTTTTTTTGCGCGGGGCACG | 55 |
| IgE | NO: 5 | TTTATCCGTCCCTCCTAGTGGCGTGCCC | |
| aptamer | | CGCGC | |
| Extended | SEQ ID | /5AzideN/TTTTTTTTTTCTGCTCGTTGGCT | 55 |
| IgE | NO: 6 | GAGGCCGTCTCGCGTCGCAGCGCTACG | |
| randomer | | GCCCC | |

### Example 4: Process Map Overview of Selective Protein Thermoprecipitation

The standard protocol for selective protein capture via thermoprecipitation begins with the incubation of the target protein in solution with a pNIPAM-aptamer conjugate. The concentration of the aptamer is generally held at a five-fold molar excess of the target protein concentration. For simple proof-of-concept studies, the pNIPAM-aptamer conjugate was incubated in binding buffer at a final concentration of approximately 1.25 mM. The binding buffer for a particular aptamer may be a solution known to mediate the proper aptamer structural conformation needed to bind to the target protein. The binding buffer is sometimes identical to the selection buffer used for aptamer panning A mixture comprising the polypeptide target, with or without a specific binding buffer, is then added so that the final concentration of aptamer is in five-fold excess to the target protein. If the target cannot be delivered in binding buffer, it is delivered in as concentrated form as possible to ensure that binding buffer dominates the solution. To determine selectivity, the target may be delivered along with an off-target protein. For challenging experiments, the binding buffer was replaced with spent CHO cell media and even CHO cell active culture media.

The target and aptamer is incubated for one hour to allow diffusion and binding in the presence of unconjugated polymer. There is no solid support/undissolved carrier present in the mixture. Unconjugated polymer is pNIPAM polymer that is not part of a polymer-aptamer conjugate complex, and is a beneficial consequence of the low conjugation chemistry yield. Unconjugated polymer helps to create discrete pellets of polymer when aptamer concentrations are low enough that the resulting conjugated polymer concentrations are too low to form pellets upon thermoprecipitation and centrifugation. After one hour of room-temperature incubation in microcentrifuge tubes, the solution is then thermoprecipitated at 40° C. and subjected to centrifugal forces (12,000 g) for thirty minutes to pull the precipitated polymer into a discrete pellet at the bottom of the tube. The pellet should contain the polymer, aptamer, and protein target captured by the aptamer. The solution above the pellet, the supernatant, should contain any uncaptured target and any off-target proteins that were present during the incubation. The supernatant, called “S1”, is removed from the pellet and set aside.

The pellet is then resuspended in chilled binding buffer at room temperature. This disperses the pellet and redissolves the polymer-aptamer conjugate in solution. Because the aptamer conformation in binding buffer should be unchanged relative to its conformation during incubation, the target protein that was initially captured should remain complexed with the polymer-aptamer conjugates upon re-dissolution and resuspension. Any proteins, on- or off-target, that were pulled down nonspecifically (via physical entrapment) during thermoprecipitation and centrifugation will also be released into solution. The solution is then subjected to a second round of thermoprecipitation and centrifugation. As with the first round, the second pellet should contain the polymer, polymer-aptamer conjugate, and protein target captured by the aptamer. The supernatant should contain proteins that may have physically entrapped in the precipitated polymer network. In this way the pellet is effectively washed. The second supernatant is called “S2”.

The S2 supernatant is removed from the pellet and set aside. The pellet is next resuspended in a dissociation solution. The nature of this solution should be mild but should mediate a conformation change in the aptamer which causes it to release the target protein into solution. When the solution is then thermoprecipitated and centrifuged, therefore, the pellet should only contain polymer and polymer-aptamer conjugate; the target protein should remain suspended in the supernatant. This third supernatant is called “D1”, because it is the first supernatant collected under dissociation conditions.

As before, the pellet is then “washed” with dissociation solution: the pellet is resuspended and redissolved in dissociation solution, and then thermoprecipitated and centrifuged again. The supernatant from this second dissociation solution wash is called “D2”. Depending on the effectiveness of the dissociation conditions on the aptamer and its subsequent release of the target protein, additional rounds of dissociation may be needed to fully release the target protein from the aptamer for complete recovery of the captured target.

### Example 5: Selective Thermoprecipitation of Human L-Selectin and Thrombin Protein

Affinity thermoprecipitation of human L-Selectin protein and Thrombin protein was investigated using 18 kDa and 28 kDa polymer-aptamer conjugates, which were prepared using aptamer or randomer sequences SEQ ID NO: 1, SEQ ID NO: 2 and SEQ ID NO: 3 according to the preceding examples. Polymer-aptamer conjugates were incubated in binding buffer containing 200 nM of L-Selectin or Thrombin protein. Final molar concentrations of conjugated aptamer were estimated to be approximately five-fold greater than the spiked target protein. TABLES 2 and 3 summarize the specific polymer-aptamer combinations that were tested by thermoprecipitation with on-target or off-target proteins:

TABLE 2
L-Selectin studies

| Series Label | | Spiked Protein | Polymer Mn | Conjugated Aptamer |
|---|---|---|---|---|
| γ | Gamma | L-Selectin | 18 kDa | selectin |
| δ | Delta | L-Selectin | 18 kDa | thrombin |
| ε | Epsilon | L-Selectin | 28 kDa | selectin |
| η | Eta | L-Selectin | 28 kDa | thrombin |
| ν | Nu | L-Selectin | 18 kDa | thrombin randomer |
| ∘ | Omicron | L-Selectin | 28 kDa | thrombin randomer |

TABLE 3
Thrombin studies

| Series Label | | Spiked Protein | Polymer Mn | Conjugated Aptamer |
|---|---|---|---|---|
| σ | Sigma | Thrombin | 18 kDa | thrombin |
| ρ | Rho | Thrombin | 18 kDa | selectin |
| ζ | Zeta | Thrombin | 28 kDa | selectin |
| θ | Theta | Thrombin | 28 kDa | thrombin |
| ξ | Xi | Thrombin | 18 kDa | thrombin randomer |
| π | Pi | Thrombin | 28 kDa | thrombin randomer |

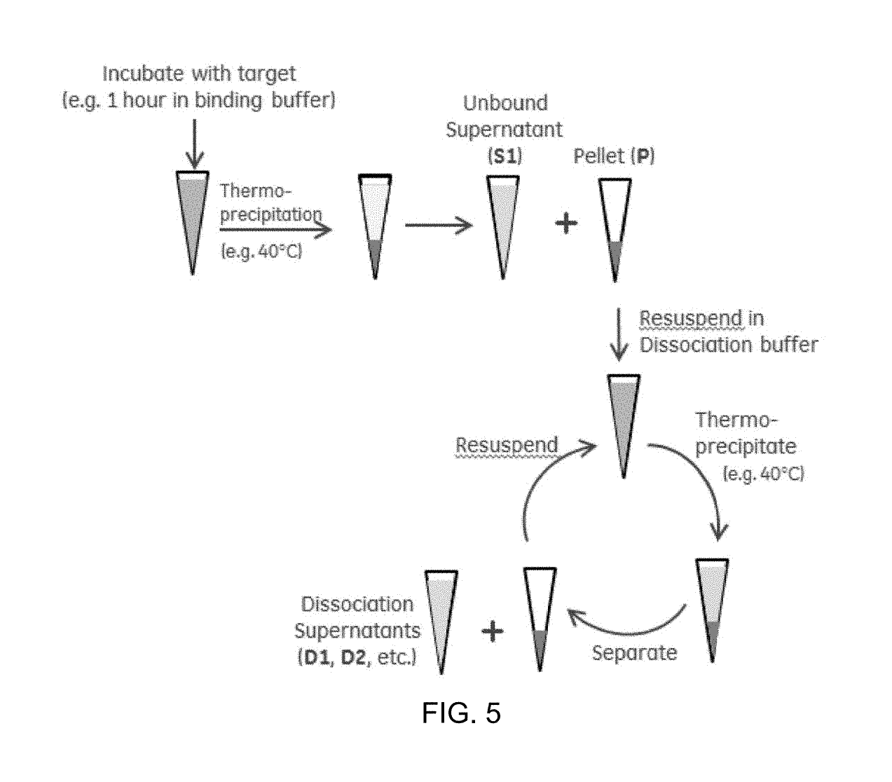

HBS-P buffer (10 mM HEPES pH 7.4, 150 mM NaCl, 0.005% v/v polysorbate 20) was used as a binding buffer in these studies. After 1 hr incubation at room temperature with on-target or off-target protein, thermoprecipitation of the polymer-aptamer conjugates was conducted as generally outlined in FIG. 5. To ensure complete and absolute polymer recovery, the binding buffer was centrifuged at 12,000×g for 30 minutes at 40° C., thereby generating a pellet fraction (P) and a supernatant fraction (S1). The S1 fraction (comprising unbound protein) was set aside for testing, while the P fraction was optionally washed in cold binding buffer or resuspended in a cold dissociation buffer. For selectin aptamer, the dissociation buffer was 1×TBE (Tris/borate/EDTA), while for thrombin aptamer the dissociation buffer was 0.5M NaCl. These specific dissociation buffers proved optimal in surface plasmon resonance studies (SPR) using biotin-labeled aptamers (immobilized onto an SPR surface) with L-Selectin or Thrombin protein in the solution phase. After incubation in dissociation buffer (e.g. 30 minutes), thermoprecipitation of the polymer-aptamer conjugates was repeated. Again, to ensure complete and absolute polymer recovery, the dissociation buffer was centrifuged at 12,000×g for 30 minutes at 40° C., thereby generating a first dissociation fraction (D1) and a pellet fraction (P). The D1 fraction (comprising bound and eluted protein) was saved while the pellet fraction was optionally resuspended in cold dissociation buffer to repeat the thermoprecipitation process for one, two, or three more rounds, thereby generating a second, third, or fourth dissocation fraction (D2, D3, D4, and so on). Equal volumes of S1 and D1 fractions were mixed with 2×Tris-Glycine SDS Sample Buffer (ThermoFisher) and analyzed by SDS-PAGE. L-Selectin and Thrombin bands were quantified by gel densitometry (Image J software) after staining the gel with SYPRO Ruby, and percent recovery was calculated as follows: [D1/(D1+S1)]×100.

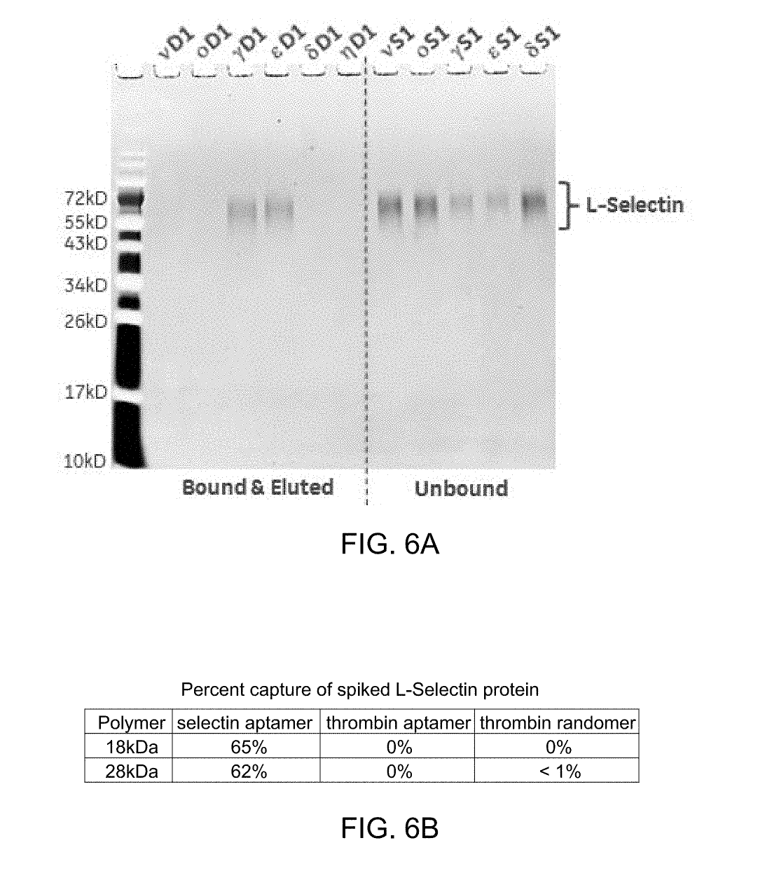

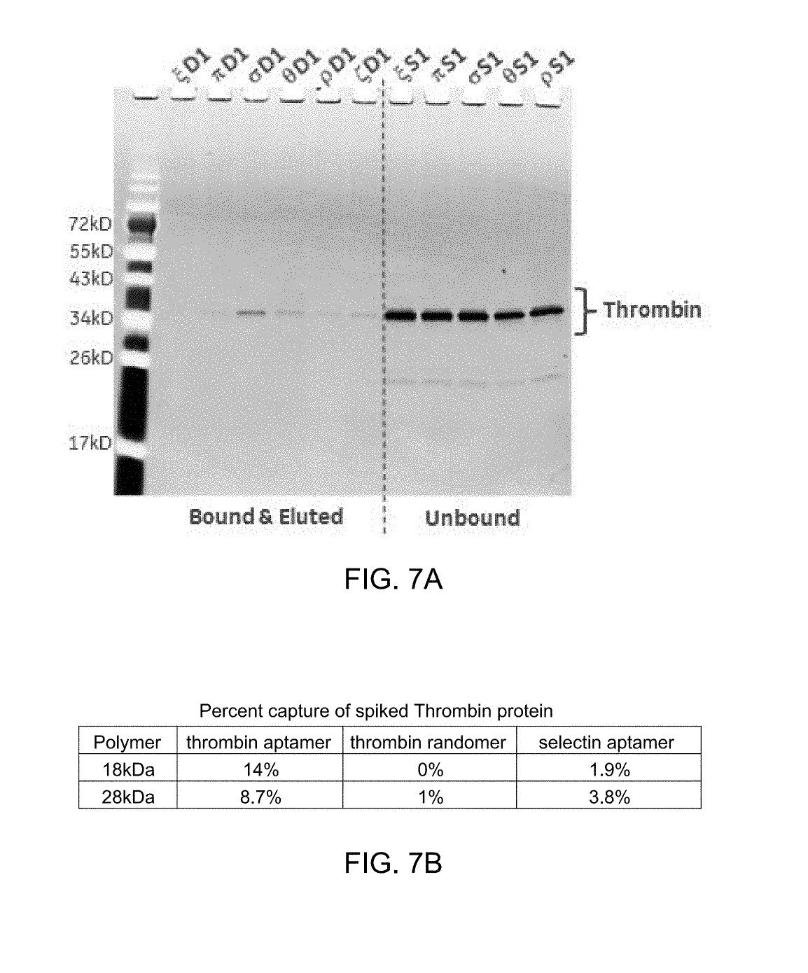

FIG. 6A and FIG. 6B and FIG. 7A and FIG. 7B depict the results of the thermoprecipitation experiments outlined in TABLES 2 and 3. In total, 6A and FIG. 6B and FIG. 7A and FIG. 7B reveal that protein recovery from polymer-aptamer thermoprecipitates is highly aptamer-dependent. The selectin aptamer exhibited high sensitivity and specificity for L-Selectin protein when conjugated to polymer and resulted in >60% recovery for on-target combinations and no measurable recovery for off-target combinations (FIG. 6A and FIG. 6B). This data confirms that protein entrapment or semi-specific association to the thermoresponsive polymer itself is very low. The thrombin aptamer proved less sensitive and specific for Thrombin protein and resulted in only modest recovery for on-target combinations with measurable off-target recovery as well (FIG. 7A and FIG. 7B). Further optimization of aptamer sequence, binding buffer, incubation time, or thermoprecipitation conditions may improve upon these results.

### Example 6: Selective Thermoprecipitation of Human IgE Antibody

Affinity thermoprecipitation of human IgE antibody was investigated using 28 kDa polymer-aptamer conjugates, which were prepared using aptamer sequences SEQ ID NO: 4, SEQ ID NO: 5 and SEQ ID NO: 6 according to the preceding examples. Polymer-aptamer conjugates were incubated in binding buffer containing 200 nM of human IgE antibody (Athens Research & Technology). Final molar concentrations of conjugated aptamer were estimated to be approximately five-fold greater than the spiked IgE antibody. TABLE 4 summarizes the specific polymer-aptamer combinations that were tested by thermoprecipitation with on-target or off-target specificity:

TABLE 4

| Series Label | Spiked Protein | Polymer Mn | Conjugated Aptamer |
|---|---|---|---|
| A | IgE | 28 kDa | randomer |
| D | IgE | 28 kDa | extended IGE |
| II | IgE | 28 kDa | IGE |

Phosphate buffered saline containing 1 mM MgCl₂ and 0.005% Tween-20 was used a binding buffer in these studies. After 1 hr incubation at room temperature, thermoprecipitation of the polymer-aptamer conjugates was conducted as generally outlined in FIG. 5. To ensure complete and absolute polymer recovery, the binding buffer was centrifuged at 12,000×g for 30 minutes at 40° C., thereby generating a pellet fraction (P) and a supernatant fraction (S1). The S1 fraction (comprising unbound antibody) was reserved, while the P fraction was optionally washed in cold binding buffer or resuspended in a cold dissociation buffer comprising 0.5M NaCl. After incubation in dissociation buffer (e.g. 30 minutes), thermoprecipitation of the polymer-aptamer conjugates was repeated. Again, to ensure complete and absolute polymer recovery, the dissociation buffer was centrifuged at 12,000×g for 30 minutes at 40° C., thereby generating a first dissociation fraction (D1) and a pellet fraction (P). The D1 fraction (comprising bound and eluted antibody) was saved while the pellet fraction was optionally resuspended in cold dissociation buffer to repeat the thermoprecipitation process for one, two, or three more rounds, thereby generating a second, third, or fourth dissocation fraction (D2, D3, D4, and so on). Equal volumes of S1 and D1 fractions were mixed with 2×Tris-Glycine SDS Sample Buffer (ThermoFisher) and analyzed by non-reducing SDS-PAGE. Full-length IgE was quantified by gel densitometry (Image J software) after staining the gel with SYPRO Ruby, and percent recovery was calculated as follows: [D1/(D1+S1)]×100.

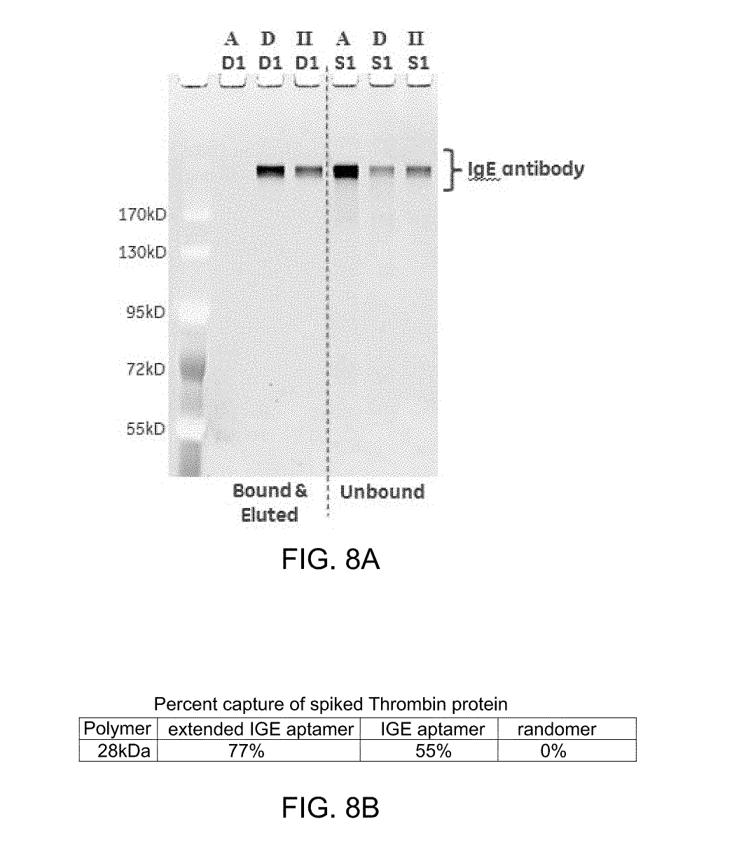

FIG. 8A and FIG. 8B depict the results of the thermoprecipitation experiments outlined in TABLE 3. In total, FIG. 8A and FIG. 8B demonstrate that antibody recovery from polymer-aptamer thermoprecipitates is highly sensitive and specific. Both IGE and extended IGE aptamer-polymer conjugates resulted in high IgE recovery from solution (approximately 55% and 77%, respectively) with no measurable non-specific recovery using a randomized aptamer sequence (FIG. 8A and FIG. 8B). This data confirms that antibody entrapment or semi-specific association to the thermoresponsive polymer itself is very low.

### Example 7: Selective Antibody Thermoprecipitation from Harvested CHO Media or Active CHO Cell Culture

Affinity thermoprecipitation of human IgE antibody was investigated using 28 kDa polymer conjugated to IGE aptamer sequence SEQ ID NO: 4 that was prepared according to the preceding examples. Polymer-aptamer conjugates were incubated in spent CHO media (harvested from a saturated culture in ActiCHO media by centrifugation) that was spiked with 200 nM of human IgE antibody (Athens Research & Technology). The final molar concentration of conjugated IGE aptamer was estimated to be approximately five-fold greater than the spiked IgE antibody. After overnight incubation in harvested CHO media at room temperature, thermoprecipitation of the polymer-aptamer conjugates was conducted as generally outlined in FIG. 5. To ensure complete and absolute polymer recovery, the CHO media was centrifuged at 12,000×g for 30 minutes at 40° C., thereby generating a pellet fraction (P) and a supernatant fraction (S1). The S1 fraction (comprising unbound antibody) was reserved, while the P fraction was optionally washed in cold binding buffer (phosphate buffered saline containing 1 mM MgCl₂ and 0.005% Tween-20) and re-thermoprecipitated, thereby generating a second supernatant fraction (S2) and a pellet fraction (P). The S2 fraction (comprising a wash fraction) was reserved, while the P fraction was resuspended in a cold dissociation buffer comprising 0.5M NaCl. After incubation in dissociation buffer (e.g. 30 minutes), thermoprecipitation of the polymer-aptamer conjugate was repeated. Again, to ensure complete and absolute polymer recovery, the dissociation buffer was centrifuged at 12,000×g for 30 minutes at 40° C., thereby generating a first dissociation fraction (D1) and a pellet fraction (P). The D1 fraction (comprising bound and eluted antibody) was saved while the pellet fraction was optionally resuspended in cold dissociation buffer to repeat the thermoprecipitation process for one, two, or three more rounds, thereby generating a second, third, or fourth dissocation fraction (D2, D3, D4, and so on). Equal volumes of S1, S2, D1, D2, and D3 fractions and eluted polymer P were mixed with 2×Tris-Glycine SDS Sample Buffer (ThermoFisher) and analyzed by non-reducing SDS-PAGE. In parallel, control samples comprising unspiked CHO media and a purified spike-equivalent of IgE antibody were analyzed by SDS-PAGE. Full-length IgE was quantified by gel densitometry (Image J software) after staining the gel with SYPRO Ruby.

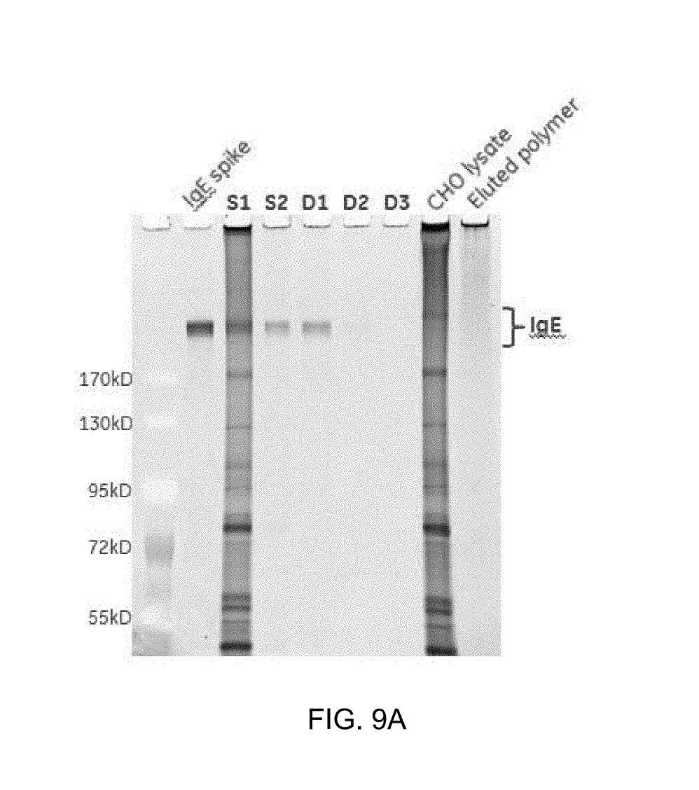

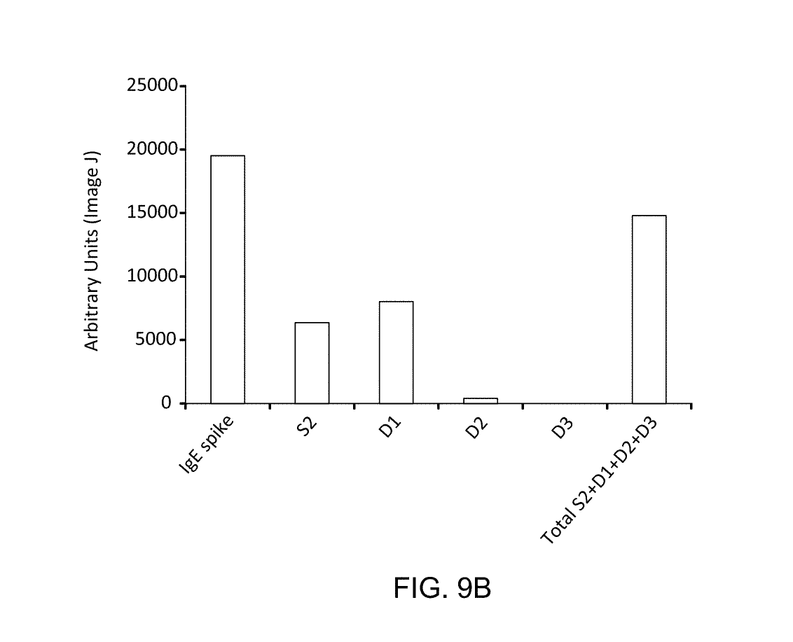

FIG. 9A depicts the results of this thermoprecipitation experiment and shows that the polymer-aptamer conjugate is sensitive and specific for antibody purification from harvested CHO media and minimizes host-cell protein (HCP) contamination. Little to no HCP is visible in S2 and D1 fractions and remained unbound is the S1 fraction (FIG. 9A). The vast majority of bound IgE was properly eluted in the D1 fraction, with little to no additional release in subsequent dissociation cycles (FIG. 9A). By summing the total IgE content of S2, D1, D2, and D3 fractions, it was revealed that approximately 75% of the original IgE spike equivalent was recovered from spent CHO media in this experiment (FIG. 9B). Further optimization of aptamer sequence, binding buffer, incubation time, or thermoprecipitation conditions may improve upon these results.

To investigate antibody thermoprecipitation in the presence of live CHO cells, the preceding experiment was repeated by incubating the polymer-aptamer conjugate in an active CHO culture (cell density was approximately 1.02×10⁶ cells/mL in Acti-CHO media) spiked with 200 nM of human IgE antibody (Athens Research & Technology). The polymer-aptamer conjugate was prepared from 28 kD polymer and an extended IGE aptamer sequence SEQ ID NO: 4 as described in the preceding examples. The final molar concentration of conjugated aptamer was estimated to be approximately five-fold greater than the spiked IgE antibody. Polymer-aptamer conjugate was incubated with active CHO culture for 1 hour at room temperature (i.e. the binding step), after which the cells were pre-cleared from the mixture with gentle centrifugation at room temperature (300×g, 10 min, 20° C.). The supernatant fraction (comprising CHO media, polymer-aptamer conjugate, and IgE antibody) was split into two aliquots to evaluate different precipitation methods. The first method was identical to the preceding experiment and utilized centrifugation at 12,000×g for 30 minutes at 40° C. to ensure complete and absolute polymer recovery. In contrast, the second method utilized a 30 minute incubation step at 40° C. to passively settle the polymer-aptamer conjugate out of solution. A course frit filter basket was used to retain the thermoprecipitate, and aqueous fractions were collected by brief centrifugation (10 sec-3 min). All S1, D1, and D2 fractions were collected using these two different thermoprecipitation methods, and equal volume were mixed with 2×Tris-Glycine SDS Sample Buffer (ThermoFisher) and analyzed by non-reducing SDS-PAGE. Full-length IgE was quantified by gel densitometry (Image J software) after staining the gel with SYPRO Ruby.

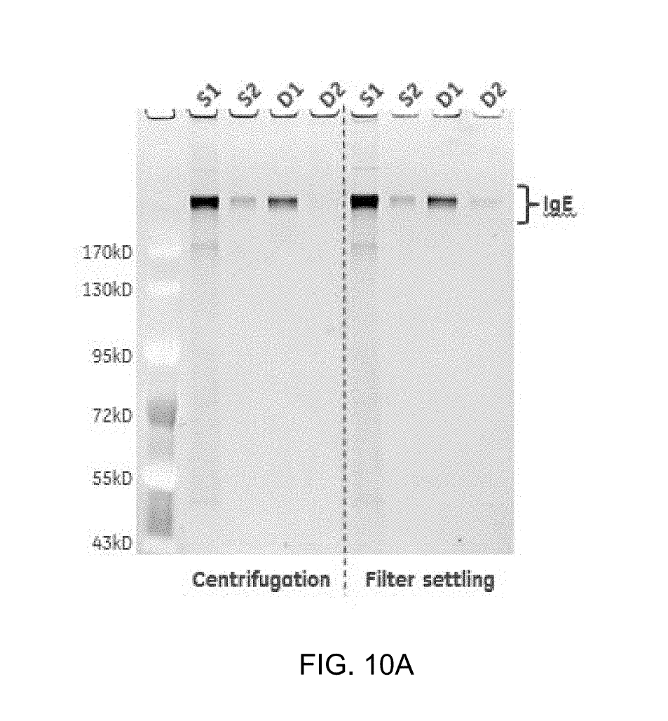

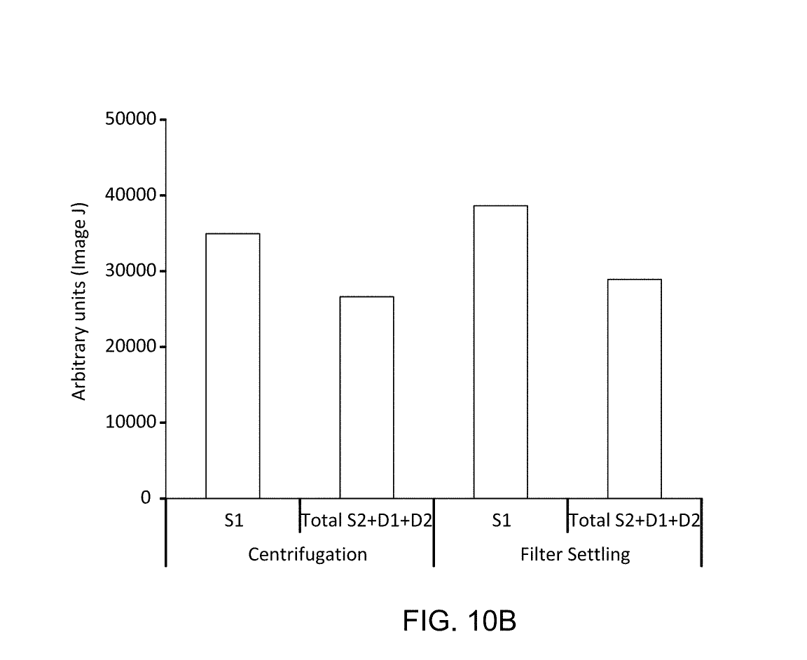

FIG. 10A depicts the results of these thermoprecipitation experiments from active CHO culture using two different processing methods. In total, FIG. 10A demonstrates that the polymer-aptamer conjugate is sensitive and specific for antibody purification even in the presence of live CHO cells and minimizes host-cell protein (HCP) contamination. Similar quantities of purified antibody are observed using either centrifugation-based processing or passive settling of the thermoprecipitate in a coarse filter basket. With a binding step of 1 hour in the presence of live cells, the total IgE content of S2, D1, and D2 fractions is similar (˜43%) to the amount of unbound antibody in the S1 fraction (˜57%) (FIG. 10B). Further optimization of aptamer sequence, binding buffer, incubation time, or thermoprecipitation conditions may improve upon these results.

### Example 8: Recycling of Polymer-Aptamer Conjugate for Continuous Use

Re-use of a polymer-aptamer conjugate was investigated using 28 kDa polymer conjugated to extended IGE aptamer sequence SEQ ID NO: 4 according to the preceding examples. Polymer-aptamer conjugates were incubated in binding buffer containing 200 nM of human IgE antibody (Athens Research & Technology) in two separate rounds, wherein the second round of binding was performed using polymer-aptamer recycled from the first round of binding. In both rounds, the final molar concentration of conjugated aptamer was estimated to be approximately five-fold greater than the spiked IgE antibody. Phosphate buffered saline containing 1 mM MgCl₂ and 0.005% Tween-20 was used a binding buffer in both rounds, and thermoprecipitation of the polymer-aptamer conjugate was conducted as generally outlined in FIG. 5 and described in detail in Example 6. Equal volumes of S1, S2, and D1 fractions from the first round (series A) and second round (series RP for Recycled Polymer) were mixed with 2×Tris-Glycine SDS Sample Buffer (ThermoFisher) and analyzed by non-reducing SDS-PAGE. Full-length IgE was quantified by gel densitometry (Image J software) after staining the gel with SYPRO Ruby, and percent recovery was calculated as follows: [(S2+D1)/(S1+S2+D1)]×100.

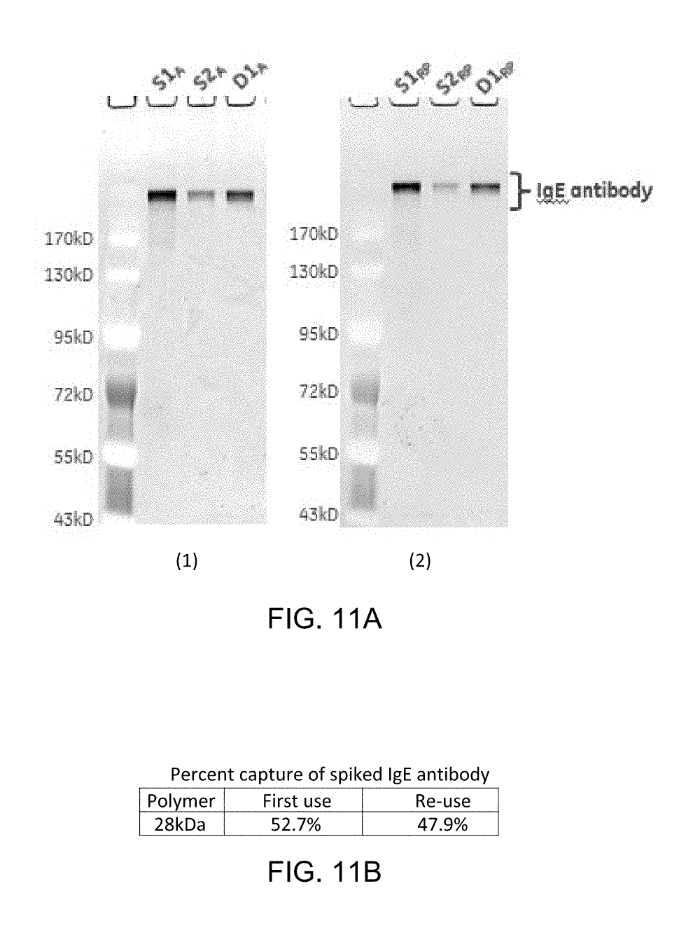

FIG. 11A and FIG. 11B depict the results of these thermoprecipitation experiments wherein the polymer-aptamer conjugate from the first round of thermoprecipitation (series A) was re-used for a second round of thermoprecipitation (series RP for Recycled Polymer). In total, FIG. 11A and FIG. 11B illustrate that the polymer-aptamer conjugate can indeed be recycled with a minimal loss of activity, as the amount of antibody recovered in the first round (˜52.7%) is similar to the amount recovered in the second round after re-using the polymer-aptamer conjugate (˜47.9%). Further optimization of aptamer sequence, binding buffer, incubation time, or thermoprecipitation conditions may improve upon these results.

While only certain features of the invention have been illustrated and described herein, many modifications and changes will occur to those skilled in the art. It is, therefore, to be understood that the appended claims are intended to cover all such modifications and changes as fall within the true spirit of the invention.

1. A method for selective purification of one or more than one untagged target polypeptide, the method comprising:
(a) contacting an aptamer-conjugated environmentally-responsive homopolymer with a mixture comprising one or more than one untagged target polypeptide; and
(b) precipitating the aptamer-conjugated environmentally-responsive homopolymer complexed to one or more than one untagged target polypeptide from the mixture by changing at least one property of the mixture;
wherein the environmentally-responsive homopolymer has a number average molecular weight greater than 11.5 kDa;
wherein steps (a) and (b) are carried out in the absence of a solid support or insoluble carrier material; and
wherein steps (a) and (b) are carried out under substantially matching pH conditions and salt concentrations.

2. The method of claim 1, wherein the environmentally-responsive homopolymer is a thermo-responsive homopolymer.

3. The method of claim 1, wherein the changing at least one property of the mixture comprises changing the temperature of the mixture such that step (a) is carried out at a first temperature and step (b) is carried out at a second temperature which is different from the first temperature.

4. The method of claim 3, wherein the first temperature is below the lower critical solution temperature (LCST) of the aptamer-conjugated thermo-responsive homopolymer and the second temperature is higher than the LCST of the aptamer-conjugated thermo-responsive homopolymer.

5. The method of claim 4, wherein the LCST of the aptamer-conjugated thermo-responsive homopolymer is less than about 45° C.

6. The method of claim 1, wherein the polypeptide comprises a protein, an antigenic peptide, a vaccine, an enzyme, antibody, a drug-conjugate, glycoprotein, a glycoprotein, or a combination thereof.

7. The method of claim 1, further comprising step (c): separating the precipitated aptamer-conjugated environmentally-responsive homopolymer complexed to one or more than one untagged target polypeptide from the mixture by filtration, gravitational settling, centrifugation, electrostatic means, ultrasonic means, or magnetic means.

8. The method of claim 7, further comprising step (d): treating the separated aptamer-conjugated environmentally-responsive homopolymer complexed to one or more than one untagged target polypeptide with a condition that induces dissociation of the aptamer-conjugated environmentally-responsive homopolymer from the one or more than one untagged target polypeptide; step (e): precipitating the dissociated aptamer-conjugated environmentally-responsive homopolymer by changing at least one property of the mixture of step (d); and step (f): isolating a selectively-purified untagged polypeptide from the supernatant of step (e).

9. The method of claim 8, further comprising step (g): reusing the precipitated aptamer-conjugated environmentally-responsive homopolymer of step (e) for selective purification of one or more untagged polypeptide in a continuous manner, wherein the precipitated aptamer-conjugated environmentally-responsive homopolymer is resuspended with a new mixture comprising one or more than one untagged target polypeptide.

10. The method of claim 8, wherein the condition that induces dissociation of the aptamer from the one or more than one untagged target polypeptide is selected from the group consisting of suspension in water, applying a chelating agent, a pH which is different from the pH of the mixture in step (a), a temperature which is different from the temperature of the mixture in than step (a) and step (b); a salt concentration which is different from the salt concentration of the mixture in step (a), or a combination thereof.

11. The method of claim 1, wherein the homopolymer is poly(N-isopropylacrylamide) (pNIPAM).

12. The method of claim 1, wherein the environmentally-responsive homopolymer has a number average molecular weight greater than 12 kDa.

13. The method of claim 1, wherein the environmentally-responsive homopolymer has a number average molecular weight greater than 15 kDa.

14. A method for selective purification of one or more than one untagged target polypeptide, the method comprising:
(a) contacting an aptamer-conjugated thermo-responsive homopolymer with a mixture comprising one or more than one untagged target polypeptide; and
(b) precipitating the one or more than one untagged target polypeptide complexed with the aptamer-conjugated thermo-responsive homopolymer from the mixture by changing the temperature of the mixture;
wherein the LCST of the aptamer-conjugated thermo-responsive homopolymer is less than about 45° C.;
wherein steps (a) and (b) are carried out in the absence of a solid support or insoluble carrier material; and
wherein steps (a) and (b) are carried out under substantially matching pH conditions and salt concentrations.

15. The method of claim 14, wherein the thermo-responsive homopolymer has a number average molecular weight greater than 11.5 kDa.

16. The method of claim 14, wherein the thermo-responsive homopolymer has a number average molecular weight greater than 15 kDa.

17. A method for selective purification of one or more than one untagged target polypeptide, the method comprising:
(a) contacting an aptamer-conjugated pNIPAM homopolymer with a mixture comprising one or more than one untagged target polypeptide;
(b) precipitating the one or more than one untagged target polypeptide complexed to the aptamer-conjugated pNIPAM homopolymer from the mixture by changing the temperature of the mixture;
(c) separating the precipitated aptamer-conjugated pNIPAM homopolymer complexed to the one or more than one untagged target polypeptide from the supernatant by filtration or gravitational settling or centrifugation;
(d) treating the separated aptamer-conjugated pNIPAM homopolymer complexed to the one or more than one untagged target polypeptide with a condition that induces dissociation of the aptamer-conjugated pNIPAM homopolymer from the one or more than one untagged target polypeptide;
(e) precipitating the dissociated aptamer-conjugated pNIPAM homopolymer by changing the temperature of the mixture of step (d);
(f) isolating the one or more than one untagged polypeptide from the supernatant of step (e); and
(g) optionally reusing the precipitated aptamer-conjugated pNIPAM homopolymer from step (e) for repeating steps (a) to (f);
wherein the pNIPAM homopolymer has a number average molecular weight greater than 11.5 kDa;
wherein steps (a) and (b) are carried out in the absence of a solid support or insoluble carrier material; and
wherein steps (a) and (b) are carried out under substantially matching pH conditions and salt concentrations.

18. The method of claim 17, wherein the condition that induces dissociation of the aptamer from the one or more than one untagged target polypeptide is selected from the group consisting of suspension in water, applying a chelating agent, a pH which is different from the pH of the mixture in step (a), a temperature which is different from the temperature of the mixture in step (a) and step (b); a salt concentration which is different from the salt concentration of the mixture in step (a), or a combination thereof.

19. The method of claim 17, wherein the aptamer-conjugated pNIPAM homopolymer has an LCST of less than about 45° C.

20. The method of claim 17, wherein the pNIPAM homopolymer has a number average molecular weight greater than 15 kDa.
